(19) United States
(12) Patent Application Publication
(10) Pub. No.: US 2023/0242611 A1
(43) Pub. Date: Aug. 3, 2023

(54) MAGE-A1 SPECIFIC T CELL RECEPTOR AND USES THEREOF

(71) Applicant: Yeda Research and Development Co. Ltd., Rehovot (IL)

(72) Inventors: Rachel Lea EISENBACH, Mazkeret Batya (IL); David BASSAN, Rehovot (IL); Yosi GOZLAN, Rehovot (IL); Esther TZEHOVAL, Rehovot (IL); Nir FRIEDMAN, Rehovot (IL); Erez GREENSTEIN, Rehovot (IL)

(73) Assignee: Yeda Research and Development Co. Ltd., Rehovot (IL)

(21) Appl. No.: 18/107,573

(22) Filed: Feb. 9, 2023

Related U.S. Application Data
(63) Continuation of application No. PCT/IL2021/050959, filed on Aug. 6, 2021.

(30) Foreign Application Priority Data
Aug. 9, 2020 (IL) 276599

(51) Int. Cl. C07K 14/725; C12N 9/78; A61K 39/00; A61P 35/00

(52) U.S. Cl. CPC C07K 14/7051; C12Y 305/04005; C12N 9/78; A61K 39/4632; A61K 39/4611; A61K 39/464486; A61P 35/00; A61K 39/4637; A61K 2239/25

(57) ABSTRACT

MAGE-A1 specific T cell receptors (TCRs) are provided. Accordingly, there is provided a TCR comprising a TCR α chain as set forth in SEQ ID NO: 1 having at least one mutation at an amino acid position selected from the group consisting of S189, G125, W55 and Y56; and/or a TCR β chain as set forth in SEQ ID NO: 2 having at least one mutation at an amino acid position selected from the group consisting of S32, S109 and T63, the TCR binds a MAGE-A1 peptide as set forth in SEQ ID NO: 25. Also provided are polynucleotides encoding the TCR and T cells expressing same and methods of use thereof.

### RELATED APPLICATIONS

This application is a Continuation of PCT Patent Application No. PCT/IL2021/050959 having International filing date of Aug. 6, 2021, which claims the benefit of priority Israel Patent Application No. 276599 filed on Aug. 9, 2020. The contents of the above applications are all incorporated by reference as if fully set forth herein in their entirety.

### SEQUENCE LISTING STATEMENT

The XML file, entitled 95161Sequence Listing.xml, created on Feb. 9, 2023, comprising 84,942 bytes, submitted concurrently with the filing of this application is incorporated herein by reference.

### FIELD AND BACKGROUND OF THE INVENTION

The present invention, in some embodiments thereof, relates to a MAGE-A1 specific T cell receptor and uses thereof.

Cancer immunotherapy, including cell-based therapy, antibody therapy and cytokine therapy, has emerged in the last couple of years as a promising strategy for treating various types of cancer owing to its potential to evade genetic and cellular mechanisms of drug resistance and to target tumor cells while sparing healthy tissues.

Cell-based therapy using native or genetically engineered T cells having a T cell receptor (TCR) specific for an antigen differentially expressed in association with an MHC class I molecule on cancer cells were shown to exert anti-tumor effects in several types of cancers.

TCR specificity and avidity (i.e. the affinity and the number of pMHC-TCR contacts) are crucial factors for the effectiveness of TCR based therapy. Basically, T cell responsiveness increases with TCR affinity. However, there is a plateau at the higher end (K_{D}=1-5 μM) of the physiological range (K_{D}=1-100 μM) (4, 5); and TCRs with affinity well above the physiological range can either lose specificity in CD8+ cells (6, 7) or have reduced activity (4, 5). In addition, since the T cell repertoire is controlled by negative and positive selection in the thymus, naturally occurring TCRs have mostly low affinities, in the range of 1-100 μM.

A number of methods have been developed to obtain TCRs with higher avidity. These methods include vaccination of mice transgenic for a human MHC molecule with a human tumor associated antigens (TAA) that is not subject to central tolerance in mice (10); performing random mutagenesis of a small region, followed by screening in phage (11), yeast (12), or T-cell display (13); isolating allo-restricted T cells from HLA-mismatched donors with high affinity to the target TAA on a non-self HLA allele; and avidity maturation using somatic hypermutation (SHM) (14 and International Patent Application Publication No. WO2012/104843). For any of these approaches cross-reactivity and recognition of low levels of antigen must be carefully examined, because the resulting TCRs were not subjected to central tolerance to the human proteome on the target HLA allele.

MAGE-A1 is an antigen known to be exclusively expressed in the testis and in a variety of malignancies, including multiple myeloma, melanoma, lung, breast, colon, and ovarian cancer (10, 21). This pattern of expression makes MAGE-A1 an attractive target for cancer immunotherapy. Indeed, at least two MAGE-A1 specific TCRs have been discovered and characterized: hT27, a low affinity variant isolated from the human CTL27 clone; and T1367, a high affinity variant isolated from mice transgenic for both human TCRαβ and HLA-A2 (10).

### ADDITIONAL BACKGROUND ART INCLUDES

US Patent Application Publication No. US 20120151613;

International Patent Application Publication No. WO 2008103475.

### SUMMARY OF THE INVENTION

According to an aspect of some embodiments of the present invention there is provided a T cell receptor (TCR) comprising a TCR α chain as set forth in SEQ ID NO: 1 having at least one mutation at an amino acid position selected from the group consisting of S189, G125, W55 and Y56; and/or a TCR β chain as set forth in SEQ ID NO: 2 having at least one mutation at an amino acid position selected from the group consisting of S32, S109 and T63, the TCR binds a MAGE-A1 peptide as set forth in SEQ ID NO: 25.

According to some embodiments of the invention, the mutation in S189 comprises an S189G, the mutation in G125 comprises a G125A or G125V, the mutation in W55 comprises a W55L, the mutation in Y56 comprises a Y56F, the mutation in S32 comprises a S32T, the mutation in S109 comprises a S109N and/or the mutation in T63 comprises a T63I.

According to some embodiments of the invention, the TCR comprises:

(i) a TCR α chain as set forth in SEQ ID NO: 1 and a TCR β chain as set forth in SEQ ID NO: 2 having S32T and S109N mutations;

(ii) a TCR α chain as set forth in SEQ ID NO: 1 and a TCR β chain as set forth in SEQ ID NO: 2 having a S109N mutation;

(iii) a TCR α chain as set forth in SEQ ID NO: 1 and a TCR β chain as set forth in SEQ ID NO: 2 having a T63I mutation;

(iv) a TCR α chain as set forth in SEQ ID NO: 1 having a S189G mutation and a TCR β chain as set forth in SEQ ID NO: 2;

(v) a TCR α chain as set forth in SEQ ID NO: 1 having a G125A mutation and a TCR β chain as set forth in SEQ ID NO: 2;

(vi) a TCR α chain as set forth in SEQ ID NO: 1 having a G125V mutation and a TCR β chain as set forth in SEQ ID NO: 2;

(vii) a TCR α chain as set forth in SEQ ID NO: 1 having W55L and Y56F mutations and a TCR β chain as set forth in SEQ ID NO: 2;

(viii) a TCR α chain as set forth in SEQ ID NO: 1 having W55L, Y56F and S189G mutations and a TCR β chain as set forth in SEQ ID NO: 2; or

(ix) a TCR α chain as set forth in SEQ ID NO: 1 having a S189G mutation and a TCR β chain as set forth in SEQ ID NO: 2 having a S109N mutation.

According to some embodiments of the invention, the TCR having increased avidity to the MAGE-A1 peptide as compared to a TCR having a TCR α chain as set forth in SEQ ID NO: 1 and a TCR β chain as set forth in SEQ ID NO: 2.

According to an aspect of some embodiments of the present invention there is provided a T cell receptor (TCR) comprising:

(i) a mutation at a constant region of a TCR α chain at an amino acid position S71 corresponding to SEQ ID NO: 38;

(ii) at least one mutation at a V region of a TCR α chain at an amino acid position selected from the group consisting of W55 and Y56 corresponding to SEQ ID NO: 39, wherein the TCR α chain comprises a TRaV5 V region;

(iii) at least one mutation at a J region of a TCR α chain at an amino acid position G12 corresponding to SEQ ID NO: 40, wherein the TCR α chain comprises a TRaJ34 J region; and/or

(iv) at least one mutation at a V region of a TCR β chain at an amino acid position selected from the group consisting of S32 and T63 corresponding to SEQ ID NO: 41, wherein the TCR β chain comprises a TRbV20-1 V region.

According to some embodiments of the invention, the mutation in S71 comprises an S71G, the mutation in G12 comprises a G12A or G12V, the mutation in W55 comprises a W55L, the mutation in Y56 comprises a Y56F, the mutation in S32 comprises a S32T and/or the mutation in T63 comprises a T63I.

According to some embodiments of the invention, the TCR binds a tumor associated antigen (TAA).

According to some embodiments of the invention, the TCR binds a MAGE-A1 peptide as set forth in SEQ ID NO: 25.

According to an aspect of some embodiments of the present invention there is provided at least one polynucleotide encoding the TCR.

According to an aspect of some embodiments of the present invention there is provided a T cell genetically engineered to express the TCR.

According to an aspect of some embodiments of the present invention there is provided method of treating cancer presenting a MAGE-A1 peptide as set forth in SEQ ID NO: 25 in a subject in need thereof, the method comprising administering to the subject a therapeutically effective amount of a T cells genetically engineered to express the TCR, thereby treating the cancer in the subject.

According to an aspect of some embodiments of the present invention there is provided T cells genetically engineered to express the TCR, for use in treating cancer presenting a MAGE-A1 peptide as set forth in SEQ ID NO: 25 in a subject in need thereof.

According to an aspect of some embodiments of the present invention there is provided a method of treating a disease that can benefit from adoptive transfer of T cells in a subject in need thereof, the method comprising administering to the subject a therapeutically effective amount of T cells genetically engineered to express the TCR, wherein pathologic cells of the subject present a peptide identified by the TCR, thereby treating the disease in the subject.

According to an aspect of some embodiments of the present invention there is provided T cells genetically engineered to express the TCR, for use in a disease that can benefit from adoptive transfer of T cells in a subject in need thereof, wherein pathologic cells of the subject present a peptide identified by the TCR.

According to some embodiments of the invention, the disease is cancer.

According to some embodiments of the invention, the cancer is selected from the group consisting of multiple myeloma, melanoma, neuroblastoma, liver, lung, breast, colon, bladder, stomach, thyroid, kidney, skin, and ovarian cancer.

According to some embodiments of the invention, the T cells are autologous to the subject.

According to an aspect of some embodiments of the present invention there is provided a method for modulating the avidity of a T cell receptor (TCR) to its ligand, the method comprising:

(a) expressing in a T cell a nucleic acid sequence encoding the TCR, the nucleic acid sequence has been codon optimized to:
(i) maximize the number of WRCH (SEQ ID NO: 5) or DGYW (SEQ ID NO: 6) nucleic acid sequences,
(ii) minimize the number of SYC or GRS nucleic acid sequences,
(iii) maximize the number of WA nucleic acid sequences and/or
(iv) minimize the number of rare codons; and

(b) expressing in the T cell Activation Induced cytidine Deaminase (AID) having an amino acid sequence as set forth in SEQ ID NO: 7.

According to an aspect of some embodiments of the present invention there is provided a method for modulating the avidity of a or a Chimeric antigen receptor (CAR) to its ligand, the method comprising:

(a) expressing in a cell a nucleic acid sequence encoding the CAR; and

(b) expressing in the cell Activation Induced cytidine Deaminase (AID).

According to some embodiments of the invention, the nucleic acid sequence expressed in the (a) has been codon optimized to:
(i) maximize the number of WRCH (SEQ ID NO: 5) or DGYW (SEQ ID NO: 6) nucleic acid sequences,
(ii) minimize the number of SYC or GRS nucleic acid sequences,
(iii) maximize the number of WA nucleic acid sequences and/or
(iv) minimize the number of rare codons.

According to some embodiments of the invention, the AID has an amino acid sequence as set forth in SEQ ID NO: 7.

According to some embodiments of the invention, the cell is a T cell.

According to some embodiments of the invention, the T cell does not express an endogenous TCR.

According to some embodiments of the invention, the T cell is BWZ.36 cell.

According to some embodiments of the invention, the method further comprising expressing CD3 in the T cell.

According to some embodiments of the invention, the nucleic acid sequence expressed in the (a) has been codon optimized to include a CAGGTG (SEQ ID NO: 27) sequence.

According to some embodiments of the invention, the expressing the AID comprises transiently expressing AID.

According to some embodiments of the invention, the ligand comprises a tumor associated antigen (TAA).

According to some embodiments of the invention, the method further comprising selecting cells expressing a TCR or a CAR with increased or decreased avidity to the ligand as compared to the TCR or the CAR prior to the expressing.

According to some embodiments of the invention, the method further comprising selecting cells expressing a TCR or a CAR with increased or decreased activity following contacting with the ligand as compared to the TCR or the CAR prior to the expressing.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A, 1B and 1C demonstrate somatic hypermutaiton (SHM)-based TCR avidity maturation system in BWZ.36 cells. FIG. 1A is a schematic representation of the SHM system in genetically engineered BWZ.36 cells, referred to herein as “BWZ-8S” cells. Text surrounded by circles represent plasmids. TetR and Tet-AID (AID under regulation by TetR) plasmids allow for inducible expression of AID. The NFAT-LacZ reporter allows for using CPRG to detect TCR signaling, which activates the transcription factor NFAT. FIG. 1B shows sequences for CDR3α (upper) and CDR3β (lower) of hT27 TCR prior to optimization and following optimization. Amino acid letter is at the beginning of the codon (3 nucleotides) for that amino acid. “Hotspot” refers to AID hotspot WRCH (SEQ ID NO: 5)/DGYW (SEQ ID NO: 6) (W=A/T, R=A/G, H=A/C/T, D=A/G/T, Y=T/C) with the location of the deamination colored in red. “Coldspot” refers to AID coldspot SYC/GRS (S=C/G, Y=C/T) with the location of the unlikely deamination colored in blue. “WA(W=A/T) hotspot” refers to hotspot of Pol η. E-box motif refers to CAGGTG (SEQ ID NO: 27) sequences. FIG. 1C demonstrates the sorting strategy for TCR avidity maturation for one (h12) of four clones. Cell were gated on live single cells. Arrows above the graph in indicate that the cells in the indicated gate were taken for an additional SHM and sorting cycle. Arrows below the graph indicate 5000 cells sorted into groups that were taken for validation and TCR sequencing.

FIG. 2 shows schematic representations of mutant hT27 TCRs identified following SHM using SMRT high throughput sequencing. Mutations, mutant number designation, and sorted groups in which the mutant TCR was identified are listed above a schematic representation of the TCR sequence. Numbers below the TCR indicate the first amino acid of the region. Length is not to scale. A star indicates the location of the mutation. Domains: C=constant, CDR=complementary determining region, FR=framework region, J=joining. Mutations α W55L and a Y56F, which alone would be designated m5 and m6, occurred as a double mutation designated m9. If a single chain is presented under the mutant TCR description, the other chain bore no mutations.

FIGS. 3A, 3B and 3C demonstrate Tetramer binding and expression levels of mutant hT27 TCRs as compared to wild type (WT) hT27 and T1367 TCRs following transduction to human PBMCs. Human PMBCs were transduced with hT27 TCRs comprising S109N mutation in the beta chain (referred to herein as “m2”), T631 mutation in the beta chain (referred to herein as “m3”), S189G mutation in the alpha chain (referred to herein as “m4”), G125V mutation in the alpha chain (referred to herein as “m8”), or W55L+Y56F mutations in the alpha chain (“m9”), or with WT hT27 and with HLA-A2-MAGE-A1₂₇₈₋₂₈₆ tetramers and anti-CD4, CD8, mTCRβ. PBMCs transduced with the high affinity T1367 TCRs were used as a reference control, Pmel-1 TCR was used as an irrelevant TCR. FIG. 3A shows geometric mean fluorescence (GMF) of tetramer staining. FIG. 3B shows percent of tetramer positive cells. FIG. 3C shows fold change compared to hT27 WT from 4 independent experiments. Error bars represent SEM. Multiple comparisons between all groups, excluding the irrelevant TCR, were performed with Tukey's correction following one-way unpaired ANOVA. A statistically significant result (α=0.05) is indicated with a shorthand representation (T=T1367, WT=WT hT27, 2=m2, 9=m9, all=all other TCRs) of the group with lower fold change above the bar of the group with higher fold change. Percent mTCR positive was determined on live CD8+ cells, and all other results were determined on live CD8+mTCRβ+ cells.

FIGS. 4A, 4B and 4C demonstrate intracellular cytokine production by human PBMCs expressing mutant hT27 TCRs following contacting with their ligand, as compared to wild type (WT) hT27 and T1367 TCRs. TCR-transduced PMBCs obtained from donor I were co-cultured with target cells for 6 hours, with BFA added 2 hours into the co-culture to prevent cytokine secretion. PBMCs transduced with the high affinity T1367 TCRs were used as a reference control, Pmel-1 TCR was used as an irrelevant TCR. EC₅₀ was calculated with non-linear regression (3-parameter) in GraphPad Prism. * indicates that minimal activity (≈0% positive cells) was not reached, and therefore the calculation is not accurate. FIG. 4A shows percent of IFNγ positive cells following co-culture with T2 cells loaded with MAGE-A1 peptide. FIG. 4B shows percent IL2 positive cells following co-culture with T2 cells loaded with MAGE-A1 peptide. FIG. 4C shows percent IFNγ positive cells following co-culture with T2 cells loaded with MAGE-A1 peptide as compared to non-specific MUC-1 peptide or 721.211-A2 (MAGE-A1+) or EL4-HHD (MAGE-A1−) cells. All assays were performed in duplicates. Cells were gated on live CD8+mTCR+ cells. Results are from 1 of 2 donors and representative of 1 of 3 independent experiments.

FIGS. 5A, 5B, 5C and 5D demonstrates the cytotoxic activity of human PBMCs expressing mutant hT27 TCRs, as compared to wild type (WT) hT27 and T1367 TCRs. TCR-transduced PBMCs were co-cultured with S³⁵-methionine labelled target cells for 5 hours at the indicated E:T ratios. PBMCs transduced with the high affinity T1367 TCRs were used as a reference control, Pmel-1 TCR was used as an irrelevant TCR. Cytotoxicity was detected using an S³⁵-methionine release assay. FIG. 5A shows cytotoxic activity towards T2 cells loaded with 10 μM MAGE-A1₂₇₈₋₂₈₆ peptide. FIG. 5B shows cytotoxic activity towards 721.211-A2 (MAGE-A1+) cells. FIG. 5C shows cytotoxic activity towards T2 cells loaded with 10 μM MUC1₁₃₋₂₁ peptide. FIG. 5D shows cytotoxic activity towards EL4-HHD (MAGE-A−) cells. Results are representative of 1 of 3 repeated experiments for (FIGS. 5A and 5C) or 2 experiments for (FIGS. 5B and 5D). Results were normalized by relative number of CD8+mTCR+ cells (determined by flow cytometry). All assays were performed in triplicates.

FIGS. 6A and 6B demonstrate screening of alanine substitution and potential peptides cross-reactive with mutant hT27 TCRs, as compared to wild type (WT) hT27 and T1367 TCRs. TCR-transduced PBMCs were c-cultured with T2 cells loaded with 10⁻⁷M of the indicated peptide. Co-culture was for 6 hours, with BFA added 2 hours into the co-culture to prevent cytokine secretion. PBMCs transduced with the high affinity T1367 TCRs were used as a reference control, Pmel-1 TCR was used as an irrelevant TCR. FIG. 6A demonstrates alanine screening—Amino acid for the substituted position is listed on the x-axis. Percent of activity (determined by IFNγ positive cells) compared to activity towards native MAGE-A A1₂₇₈₋₂₈₆ peptide is presented on the y-axis (maximum activity capped at 100%). FIG. 6B demonstrates reactivity to MAGE-A1 or several potential cross-reactive peptides containing the xxLEYVxKx (SEQ ID NO: 36) motif, xxLEYxxxx (SEQ ID NO: 35) motif, or other highly similar peptides. The peptide sequence is listed under the gene with bold letters representing amino acids share with MAGE-A1₂₇₈₋₂₈₆. All assays were performed in duplicates.

FIGS. 7A, 7B and 7C demonstrate the structural model of the hT27 TCR with simulated mutations. FIGS. 7A-B show the structural model of the variable regions built using TCRmodel. Colors: Alpha chain—red, CDR1α—pink, CDR2α—light blue, CDR3α—yellow, Beta chain—blue, CDR1β—light green, CDR2β—orange, CDR3β—purple. MHC (green) and peptide (dark purple) are from the 2YPL (PDB) structure, which contains HLA-B*5703 MHC-I, KF11 peptide from HIV, and the AGA1 TCR. Mutations: m2(βS102N on sequence, numbered as β107 on model) in green; m3 (βT63I on sequence, numbered as β57 on model) in cyan; m8 (αG125V on sequence, numbered as α142 on model) in turquoise; m9 (αW55L+Y56F on sequence, numbered as α43+44 on model) in grey and brown, respectively. FIG. 7A is a rotated view to highlight m2 and m3 on the beta chain. FIG. 7B is a rotated view to highlight m8 and m9 on the alpha chain. FIG. 7C shows the structural model of the constant regions of hT27 TCR from a mouse TCR. The structure of the 2C TCR (PDB ID: 1TCR) was used to visualize the mutation m4. Colors: Alpha chain: variable region—green, constant region—light grey, DE loop—light blue, mutation m4 (αS189G on hT27 TCR, corresponding to αS175G on 2C TCR) in red. Beta chain: variable region—orange, constant region—magenta.

FIG. 8 demonstrates dox-dependent AID expression following transduction of TetR. BWZ-8S cells were initially generated by transducing BWZ.36-CD8a cells with CD3, followed by electroporation of TetR and selection using blasticidin, followed by electroporation of Tet-inducible AID mut7.3 and selection using zeocin. BWZ-8S cells were then transduced with TetR to boost expression and ensure that AID expression is dox-dependent. PCR for AID (571 bp product), TetR (331 bp product), and mGAPDH (housekeeping gene, 73 bp product) was performed following RT-PCR of mRNA from cells cultured with or without 1 μg/ml dox.

FIG. 9 demonstrates SHM and sorting cycles of hT27 TCR-transduced BWZ-8S lines. hT27 TCR-transduced BWZ-8S lines h5, h7, h8, and h12 were incubated without dox (top row) or with dox (second row) for 24 days. Cells were stained with MAGE-A1₂₇₈₋₂₈₆ tetramers and anti-TCRβ and Gated on live TCR+ cells. Cells with a high tetramer/TCR in cycle 1 (second row) staining ratio were sorted and incubated with dox for 2 weeks. Cells with high-avidity (HA) TCRs from cycle 2 (third row), were sorted an incubated with dox for 2 weeks. 5000 cells from the following groups were sorted for further analysis and TCR sequencing: Medium-high avidity (MHA) from cycle 2 (third row), HA from cycle 3 (bottom row), and Cells with high-avidity TCRs and high TCR expression (HA HiEx) from cycle 3 (bottom row). The HA population included HA HiEx cells.

FIGS. 10A, 10B, 10C and 10D show tetramer binding curves of sorted groups following SHM of hT27 TCR-transduced BWZ-8S lines. Cell of clones h5 (FIG. 10A), h7 (FIG. 10B), h8 (FIG. 10C) and h12 (FIG. 10D) were stained with MAGE-A1₂₇₈₋₂₈₆ tetramers in concentrations of 50, 10, 1, 0.1, or 0.01 nM (concentrations in respect to monomers) and gated on live cells. Geometric mean of fluorescent intensity (GMF) of tetramer binding is on the y-axis. Non-linear regression was used to fit curves and calculate the EC₅₀.

FIG. 11 shows the results of Sanger sequencing demonstrating that nearly all cells in the h12 HA group bear a S109N mutation in the beta chain of the hT27 TCR. Chromatogram of TCR sequence from h12 HA group was analyzed by Sanger sequencing and aligned to hT27 WT as reference using Sequencer software. At base 326 only a peak for A is visible, whereas the WT reference gene is G. A mutation of G326A at the DNA level leads to S109N (m2) replacement at the protein level.

FIG. 12 demonstrates IFNγ secretion by primary mouse T cells transduced with mutant hT27 TCRs in three different assays. Primary mouse splenocytes were genetically engineered to express hT27 TCR comprising the indicated mutations and co-cultured with peptide-loaded T2 cells for 6 hours, with BFA added 2 hours into the co-culture to prevent cytokine secretion, and stained for intracellular IFNγ. Pmel-1 TCR was used as an irrelevant TCR; and Muc-1 as an irrelevant peptide. T cells expressing the low affinity WT hT27 or the high affinity T1367 TCR were used as reference control. All assays were performed in triplicates. Cell were gated on live CD8+mTCR+ cells. Results are representative of 3 of 5 independent assays.

FIGS. 13A and 13B demonstrate MAGE-A1 expression in cell lines. PCR for MAGE-A1 (324 bp product), and hGAPDH (housekeeping gene, 110 bp product) was performed following RT-PCR of mRNA from cell lines. FIG. 13A shows expression in 721.221-A2 cells. FIG. 13B shows expression in DLD-1 cells cultured with or without 1 μM 5-aza-2′-deoxycytidine (DAC). An unknown product of approximately 150 bp was observed.

FIGS. 14A, 14B, 14C and 14D demonstrate intracellular cytokine production by human PBMCs expressing mutant hT27 TCRs following contacting with their ligand, as compared to wild type (WT) hT27 and T1367 TCRs. TCR-transduced PMBCs obtained from donor II were co-cultured with target cells for 6 hours, with BFA added 2 hours into the co-culture to prevent cytokine secretion. PBMCs transduced with the high affinity T1367 TCRs were used as a reference control, Pmel-1 TCR was used as an irrelevant TCR. EC₅₀ was calculated with non-linear regression (3-parameter) in GraphPad Prism. * indicates that minimal activity (≈0% positive cells) was not reached, and therefore the calculation is not accurate. FIG. 14A shows percent of IFNγ positive cells following co-culture with T2 cells loaded with MAGE-A1 peptide. FIG. 14B shows percent IL2 positive cells following co-culture with T2 cells loaded with MAGE-A1 peptide. FIGS. 14C-D show percent IFNγ positive cells following co-culture with T2 cells loaded with MAGE-A1 peptide as compared to non-specific MUC-1 peptide or 721.211-A2 (MAGE-A1+) or EL4-HHD (MAGE-A1−) cells. All assays were performed in duplicates. Cells were gated on live CD8+mTCR+ cells (FIGS. 14A-C) or live mTCR+CD4+ cells (FIG. 14D. Results are from 1 of 2 donors and representative of 1 of 3 independent experiments.

### DESCRIPTION OF SPECIFIC EMBODIMENTS OF THE INVENTION

The present invention, in some embodiments thereof, relates to a MAGE-A1 specific T cell receptor and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cell-based therapy using native or genetically engineered T cells having a T cell receptor (TCR) specific for an antigen differentially expressed in association with an MHC class I molecule on cancer cells were shown to exert anti-tumor effects in several types of cancers. Two of the crucial factors for the effectiveness of TCR based therapy are TCR specificity and avidity (i.e. the affinity and the number of pMHC-TCR contacts).

MAGE-A1 is an antigen known to be exclusively expressed in the testis and in a variety of malignancies, including multiple myeloma, melanoma, lung, breast, colon, and ovarian cancer (10, 21).

Whilst reducing specific embodiments of the present invention to practice the present inventors have used somatic hypermutation (SHM) to induce mutations in the low affinity MAGE-A1 specific TCR, hT27. Using this methodology the present inventors were able to generate novel MAGE-A1 specific TCRs having improved avidity and activity manifested by increased binding affinity, higher production of cytokines and cytotoxic activity of T cells genetically engineered to express mutant hT27 TCRs having the identified mutations as compared to the wild-type hT27 TCR (Examples 1-3 of the Examples section which follows).

Consequently, specific embodiments of the present teachings suggest T cells expressing TCRs having these novel mutations; and their use in adoptive T cells therapy.

Thus, according to a first aspect of the present invention, there is provided a T cell receptor (TCR) comprising a TCR α chain as set forth in SEQ ID NO: 1 having at least one mutation at an amino acid position selected from the group consisting of S189, G125, G125, W55 and Y56; and/or a TCR β chain as set forth in SEQ ID NO: 2 having at least one mutation at an amino acid position selected from the group consisting of S32, S109 and T63, the TCR binds a MAGE-A1 peptide as set forth in SEQ ID NO: 25.

According to an additional or an alternative aspect of the present invention, there is provided a T cell receptor (TCR) comprising:

(i) a mutation at a constant region of a TCR α chain at an amino acid position S71 corresponding to SEQ ID NO: 38;

(ii) at least one mutation at a V region of a TCR α chain at an amino acid position selected from the group consisting of W55 and Y56 corresponding to SEQ ID NO: 39, wherein said TCR α chain comprises a TRaV5 V region;

(iii) at least one mutation at a J region of a TCR α chain at an amino acid position G12 corresponding to SEQ ID NO: 40, wherein said TCR α chain comprises a TRaJ34 J region; and/or

(iv) at least one mutation at a V region of a TCR β chain at an amino acid position selected from the group consisting of S32 and T63 corresponding to SEQ ID NO: 41, wherein said TCR β chain comprises a TRbV20 V region.

As used herein, the term “T cell receptor (TCR)” refers to a heterodimer comprising an amino acid sequence of a TCR α chain and an amino acid sequence of a TCR β chain which is capable of binding a fragment of an antigen as a peptide presented in the context of a major histocompatibility complex (MHC) molecule.

Full length TCR α and β chains comprise extracellular variable (V), joining (J) and constant (C) regions, and the β chain also usually contains a short diversity (D) region between the V and J regions (but this D region is often considered as part of the J region); a transmembrane region, and a short cytoplasmic tail at the C-terminal end. Each V region comprises three hypervariable Complementarity Determining Regions (CDRs) embedded in a framework sequence; CDR3 is believed to be the main mediator of antigen recognition. The identity of the amino acid residues in a particular TCR α and β chains that make up the disclosed regions can be determined using methods well known in the art and include, but not limited to, the International Immunogenetics (IMGT) TCR nomenclature. The unique sequences defined by the IMGT nomenclature are widely known and accessible to those working in the TCR field and can be found in the IMGT public database.

In native TCRs, the gene pools that encode the TCR α and β chains are located on different chromosomes and contain separate V, D, J and C gene segments, which are brought together by rearrangement during T cell development. This leads to a very high diversity of T cell α and β chains due to the large number of potential recombination events that occur between the 54 TCR α V genes and 61 α J genes or between the 67 β V genes, two β D genes and 13 beta J genes. The recombination process is not precise and introduces further diversity within the CDR3 region.

While specific embodiments encompass non-naturally occurring TCRs, the amino acid sequences of the TCR α and β chain may comprise any known V, D, J and/or C regions.

Non-limiting Examples of TCR α V regions include TRaV5, TRaV21, TRaV12-2.

According to specific embodiments, the TCR α V region comprises TRaV5, such as provided for example in SEQ ID NO: 39.

Non-limiting Examples of TCR α J regions include TRaJ34, TRaJ14, TRaJ20.

According to specific embodiments, the TCR α J region comprises TRaJ34, such as provided for example in SEQ ID NO: 40 or 51.

According to specific embodiments, the TCR α C region comprises the mouse TCR α C region, such as provided for example in SEQ ID NO: 38.

According to specific embodiments, the TCR α C region comprises the human TCR α C region, such as provided for example in SEQ ID NO: 52.

Non-limiting Examples of TCR β V region include TRbV20-1, TRbV28, TRbV6-5.

According to specific embodiment, the TCR β V region comprises TRaV20-1, such as provided for example in SEQ ID NO: 41.

Non-limiting Examples of TCR β D region include TRbD1, TRbD2.

According to specific embodiment, the TCR β D region comprises SEQ ID NO: 42.

Non-limiting Examples of TCR β J region include TRbJ2-7, TRbJ1-2, TRbJ2-4.

According to specific embodiment, the TCR β J region comprises TRbJ2-7, such as provided for example in SEQ ID NO: 43 or 53.

Non-limiting Examples of TCR β C regions include TRbC1 and TRbC2.

According to specific embodiments, the TCR α C region comprises the mouse TRbC2, such as provided for example in SEQ ID NO: 44.

As used herein, the terms “amino acid sequence of a TCR α chain” and “amino acid sequence of a β chain” refers to full-length polypeptides, functional fragments thereof or homologs thereof which maintain at least the ability to form an αβ heterodimer and bind a peptide presented in the context of MHC. For example, according to specific embodiments, the amino acid sequences of the α and/or β chains comprise substitution, addition and deletion mutations as further described hereinabove and below.

According to specific embodiments, the amino acid sequence of a TCR α chain and/or the amino acid sequence of a β chain comprises an extracellular domain of the TCR α chain and/or the β chain.

According to specific embodiments, the TCR is human TCR.

However, it will be appreciated that according to specific embodiments, the amino acids sequences of the α and/or β chains may be chimeric subunits that comprise, for example, the V, D, and J regions from one organism and the constant regions from a different organism.

Thus, according to specific embodiments, the V, D, and J regions are of human origin and the constant regions are of mouse origin.

Non-limiting examples of antigens encompassed by specific embodiments of the present invention are disclosed for example in International Patent Application Publication No. WO2016/199140, the contents of which are incorporated herein by reference.

According to specific embodiments, the antigen is a tumor associated antigen (TAA).

As used herein, the term “tumor associated antigen (TAA)” refers to an antigen overexpressed or solely expressed by a cancerous cell as compared to a non-cancerous cell. A TAA may be a known cancer antigen or a new specific antigen that develops in a cancer cell (i.e. neoantigens).

Non-limiting examples for known TAAa include MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-Cl/CT7, MAGE-C2, NY-ESO-1, LAGE-1, SSX-1, SSX-2(HOM-MEL-40), SSX-3, SSX-4, SSX-5, SCP-1 and XAGE, melanocyte differentiation antigens, p53, ras, CEA, MUCI, PMSA, PSA, tyrosinase, Melan-A, MART-I, gplOO, gp75, alphaactinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, plSOerbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, 0250, Ga733 (EpCAM), HTgp-175, M344, MA-50, MG7-Ag, MOV18, NB\170K, NYCO-I, RCASI, SDCCAG16, TA-90 (Mac-2 binding protein\cyclophilin C-associated protein), TAAL6, TAG72, TLP, TPS, tyrosinase related proteins, TRP-1, or TRP-2.

Other TAAs that may be expressed are well-known in the art (see for example WO00/20581; Cancer Vaccines and Immunotherapy (2000) Eds Stern, Beverley and Carroll, Cambridge University Press, Cambridge). The sequences of these tumor antigens are readily available from public databases but are also found in WO 1992/020356 AI, WO 1994/005304 AI, WO 1994/023031 AI, WO 1995/020974 AI, WO 1995/023874 AI & WO 1996/026214 AI.

According to specific embodiments, the TCR binds MAGE-A1.

According to specific embodiments, the TCR binds a MAGE-A1 peptide as set forth in SEQ ID NO: 25.

According to specific embodiments, the TCR has a selective binding to a specific peptide.

As used herein, the term “selective binding” refers to the ability to bind a specific peptide and not a peptide having a different amino acid sequence, which may be manifested as higher affinity (e.g., K_{d}) to the specific peptide (e.g. MAGE-A1 peptide as set forth in SEQ ID NO: 25) as compared to the other peptides (e.g. MUC-1 peptide as set forth in SEQ ID NO: 26).

Higher affinity can be, for example, of at least 5, 10, 100, 1000 or 10000 fold.

Methods of determining binding of the TCR to the peptide are well known in the art and include BiaCore, HPLC, Surface Plasmon Resonance assay (SPR) and flow cytometry (FACS). A non-limiting example of a specific method of determining binding of a TCR to an MHC molecule presenting a peptide is a tetramer staining assay (Ogg and McMichael, 1998). Briefly, the tetramer is a complex of four monomers. Each monomer formed from a MHC-class I molecule (e.g., HLA-2A) presenting a peptide (e.g., the MAGE-A1 peptide as set forth in SEQ ID NO: 25). The skilled artisan will understand that the staining assay may be designed using other oligomers (instead of tetramer), for instance, pentamers, hexamers, hepatmers, octamers nonamer or decamers. According to specific embodiments, the MHC-I molecule is conjugated to a biotin molecule. The tetramers are assembled by linking four biotin conjugated monomers to one molecule of APC-conjugated Streptavidin. Following, TCR expressing cells are stained with the tetramers and analyzed (e.g., for TCR antigen binding) by FACS.

According to specific embodiments, the TCR has a dissociation constant (Kd) lower than 1 μM to the peptide (e.g. MAGE-A1 peptide as set forth in SEQ ID NO: 25)

As noted, a TCR is capable of binding a peptide when is presented by (or bound to) an MHC molecule.

As used herein, the phrase “major histocompatibility complex (MHC)” refers to a complex of antigens encoded by a group of linked loci that plays a role in control of the cellular interactions responsible for physiologic immune responses, which are collectively termed H-2 in the mouse and “human leukocyte antigen (HLA)” in humans. The two principal classes of the MHC antigens, class I and class II, each comprise a set of cell surface glycoproteins which play a role in determining tissue type and transplant compatibility. According to a specific embodiment, the MHC is a human MHC (i.e. HLA).

According to a specific embodiment, the MHC is a MHC class I.

According to a specific embodiment, the MHC is HLA class I.

MHC class I molecules are expressed on the surface of nearly all cells. These molecules function in presenting peptides which are mainly derived from endogenously synthesized proteins to CD8+ T cells via an interaction with the TCR. The class I MHC molecule is a heterodimer composed of a 46-kDa heavy chain which is non-covalently associated with the 12-kDa light chain β-2 microglobulin. In humans, there are several MHC haplotypes, such as, but not limited to HLA-A2, HLA-A1, HLA-A3, HLA-A24, HLA-A26, HLA-A28, HLA-A31, HLA-A33, HLA-A34, HLA-A0201, HLA-B7, HLA-B27 and HLA-B45, their sequences can be found for example at the kabbat data base, at htexttransferprotocol://immuno.bme.nwu.edu. Further information concerning MHC haplotypes can be found in Paul, B. Fundamental Immunology Lippincott-Rven Press.

According to specific embodiments, the MHC haplotype comprises an HLA-A2 haplotype.

According to specific embodiments, the MHC haplotype comprises a haplotype selected from the group consisting of HLA-A*02:01, HLA-A*02:07, HLA-A*0.2:08 and HLA-A*0.2:12. DB-I was just giving a few random examples of HLA-A*02 members, but there are alleles from HLA-A*02:01 to HLA-A*02:939. I think the most popular ones are *02:01, *02:02, *02:03, *02:05, *02:06, *02:07, and *02:11

According to specific embodiments, the MHC haplotype comprises an HLA-A*02:01 haplotype.

According to specific embodiments, the TCR binds the peptide in an MHC-restricted manner (i.e. does not bind the MHC in an absence of the peptide, and does not bind the peptide in an absence of the MHC).

According to a specific embodiment, the TCR is capable of binding the MHC presented peptide when naturally presented on cells.

Further, full length TCR α and β chains are capable of forming a heterodimer and associate with CD3 and CD3zeta to form a TCR complex. This complex is stabilized by interactions between the transmembrane domain of the TCR chains and CD3 and CDRzeta subunits. The interaction of the TCR expressed on the surface of a T cell with a specific peptide presented by MHC induces a conformational change in the TCR that triggers phosphorylation of the ITAM domains in the CD3 and CD3zeta and transmission of an activating signal.

Hence, according to specific embodiments, the amino acid sequences of the α and β chains maintain the ability of the full length polypeptides to form a complex with CD3 and CD3-zeta and transmit an activating signal in a T cell expressing same following binding to the specific peptide.

As used herein, the term “CD3” refers to the polypeptide of the CD3G, CD3D or CD3E gene (Gene ID 917, 915, 916, respectively), and includes CD3γ, CD3δ and CD3ε.

According to specific embodiments, CD3 is human CD3.

According to a specific embodiment, the CD3 refers to the human CD3γ polypeptide, such as provided in the following Accession No. NP_000064 (SEQ ID NO: 45).

According to a specific embodiment, the CD3 refers to the human CD3δ, such as provided in the following Accession Nos. NP_000723 or NP_001035741 (SEQ ID NO: 46-47).

According to a specific embodiment, the CD3 refers to the human CD3ε, such as provided in the following Accession No. NP_000724 (SEQ ID NO: 48).

As used herein the term “CD3zeta” also known as TCRzeta or CD247 refers to the polypeptide expression product of the CD247 gene (Gene ID 919). According to a specific embodiment, the CD3zeta protein refers to the human protein, such as provided in the following GenBank Numbers NP_000725 or NP_932170 (SEQ ID NO: 49-50).

As used herein the phrase “activating signal” refers to the ability of transmitting a primary stimulatory signal resulting in cellular proliferation, maturation, cytokine production and/or induction of regulatory or effector functions.

Methods of determining signaling of an activating signal are well known in the art and include, but are not limited to, binding assay using e.g. BiaCore, HPLC or flow cytometry, enzymatic activity assays such as kinase activity assays, and expression of molecules involved in the signaling cascade using e.g. PCR, Western blot, immunoprecipitation and immunohistochemistry. Additionally or alternatively, determining transmission of a signal can be effected by evaluating T cell activation or function. Methods of evaluating T cell activation or function are well known in the art and include, but are not limited to, proliferation assays such as BRDU and thymidine incorporation, cytotoxicity assays such as chromium release, cytokine secretion assays such as intracellular cytokine staining ELISPOT and ELISA, expression of activation markers such as CD25, CD69 and CD69 using flow cytometry.

The term “amino acid sequence of TCR α and/or β chain” also encompasses functional homologues (naturally occurring or synthetically/recombinantly produced), which exhibit the desired activity (i.e., at least the able to form an αβ heterodimer and bind a peptide presented in the context of MHC). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93% , at least 94%, at least 95%, at least 96%, at least 97%, at least 98%, at least 99% or 100% identical or homologous to the amino acid sequences of the TCR α and/or β chains and/or the V, D, J and/or C regions comprised therein that are described herein; or at least 70%, at least 75%, at least 80%, at least 81%, at least 82%, at least 83%, at least 84%, at least 85%, at least 86% , at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

As used herein, “identity” or “sequence identity” refers to global identity, i.e., an identity over the entire amino acid or nucleic acid sequences disclosed herein and not over portions thereof.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the amino acid sequence of TCR α and/or β chains may comprise conservative and/or non-conservative amino acid substitutions (also referred to herein as “mutations”).

According to specific embodiments, the amino acid sequence of TCR α and/or β chains may comprise conservative substitution(s).

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with an amino acid which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

When affecting conservative substitutions the substituting amino acid should have the same or a similar functional group in the side chain as the original amino acid.

Conservative substitution tables providing functionally similar amino acids are well known in the art. Guidance concerning which amino acid changes are likely to be phenotypically silent can also be found in Bowie et al., 1990, Science 247: 1306 1310. Such conservatively modified variants are in addition to and do not exclude polymorphic variants, interspecies homologs, and alleles. Typical conservative substitutions include but are not limited to: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Amino acids can be substituted based upon properties associated with side chains, for example, amino acids with polar side chains may be substituted, for example, Serine (S) and Threonine (T); amino acids based on the electrical charge of a side chains, for example, Arginine (R) and Histidine (H); and amino acids that have hydrophobic side chains, for example, Valine (V) and Leucine (L). As indicated, changes are typically of a minor nature, such as conservative amino acid substitutions that do not significantly affect the folding or activity of the protein.

According to specific embodiments, the amino acid sequence of TCR α and/or β chains may comprise non-conservative substitution(s).

The phrase “non-conservative substitutions” as used herein refers to replacement of the amino acid as present in the parent sequence by another amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOH]—CO— for aspartic acid. Those non-conservative substitutions which fall under the scope of the present invention are those which still constitute an amino acid sequence capable of binding the specific antigen.

The TCR of some embodiments of the present invention comprises at least one of the following mutations:

(i) a mutation at a constant region of a TCR α chain at an amino acid position S71 corresponding to SEQ ID NO: 38;

(ii) at least one mutation at a V region of a TCR α chain at an amino acid position selected from the group consisting of W55 and Y56 corresponding to SEQ ID NO: 39, wherein said TCR α chain comprises a TRaV5 V region;

(iii) at least one mutation at a J region of a TCR α chain at an amino acid position G12 corresponding to SEQ ID NO: 40, wherein said TCR α chain comprises a TRaJ34 J region; and/or

(iv) at least one mutation at a V region of a TCR β chain at an amino acid position selected from the group consisting of S32 and T63 corresponding to SEQ ID NO: 41, wherein said TCR β chain comprises a TRbV20-1 V region.

According to specific embodiments, the TCR comprises one of the disclosed mutations.

According to specific embodiments, the TCR comprises at least two of the disclosed mutations.

As used herein, the phrase “corresponding to SEQ ID NO: 38”, intends to include the corresponding amino acid residue relative to any other amino acid sequence of a constant region of a TCR α chain amino acid sequence.

As used herein, the phrase “corresponding to SEQ ID NO: 39”, intends to include the corresponding amino acid residue relative to any other amino acid sequence of a TRaV5 V region of a TCR α chain amino acid sequence.

As used herein, the phrase “corresponding to SEQ ID NO: 40”, intends to include the corresponding amino acid residue relative to any other amino acid sequence of a TRaJ34 J region of a TCR α chain amino acid sequence.

As used herein, the phrase “corresponding to SEQ ID NO: 41”, intends to include the corresponding amino acid residue relative to any other amino acid sequence of a TRbV20 V region of a TCR β chain amino acid sequence.

According to specific embodiments, the mutation comprises a conservative substitution.

According to other specific embodiments, the mutation comprises a non-conservative substitution.

According to specific embodiments, the mutation is a non-naturally occurring.

According to specific embodiments, the mutation in S71 corresponding to SEQ ID NO: 38 comprises an S71G.

According to specific embodiments, the mutation in W55 corresponding to SEQ ID NO: 39 comprises a W55L.

According to specific embodiments, the mutation in Y56 corresponding to SEQ ID NO: 39 comprises a Y56F.

According to specific embodiments, the mutation in G12 corresponding to SEQ ID NO: 40 comprises a G12A or G12V.

According to specific embodiments, the mutation in S32 corresponding to SEQ ID NO: 41 comprises a S32T.

According to specific embodiments, the mutation in T63 corresponding to SEQ ID NO: 341 comprises a T63I.

According to specific embodiments, the TCR comprises a TCR α chain as set forth in SEQ ID NO: 1 having at least one of the mutations recited in (i)-(iii) hereinabove and/or a TCR β chain as set forth in SEQ ID NO: 2 having at least one of the mutation recited in (iv) hereinabove.

According to specific embodiments, the TCR comprises a TCR α chain as set forth in SEQ ID NO: 1 having at least one mutation at an amino acid position selected from the group consisting of S189, G125, W55 and Y56.

According to specific embodiments, the TCR comprises a TCR β chain as set forth in SEQ ID NO: 2 having at least one mutation at an amino acid position selected from the group consisting of S32, S109 and T63.

According to specific embodiments, the TCR comprises a TCR α chain as set forth in SEQ ID NO: 1 having at least one mutation at an amino acid position selected from the group consisting of S189, G125, W55 and Y56 and/or a TCR β chain as set forth in SEQ ID NO: 2 having at least one mutation at an amino acid position selected from the group consisting of S32, S109 and T63.

According to specific embodiments, the mutation in S189 of SEQ ID NO: 1 comprises an S189G.

According to specific embodiments, the mutation in G125 of SEQ ID NO: 1 comprises a G125A or G125V.

According to specific embodiments, the mutation in W55 of SEQ ID NO: 1 comprises a W55L.

According to specific embodiments, the mutation in Y56 of SEQ ID NO: 1 comprises a Y56F.

According to specific embodiments, the mutation in S32 of SEQ ID NO: 2 comprises a S32T.

According to specific embodiments, the mutation in S109 of SEQ ID NO: 2 comprises a S109N.

According to specific embodiments, the mutation in T63 of SEQ ID NO: 2 comprises a T63I.

According to a specific embodiment, the TCR comprises a TCR α chain as set forth in SEQ ID NO: 1 and a TCR β chain as set forth in SEQ ID NO: 2 having S32T and S109N mutations.

According to a specific embodiment, the TCR comprises a TCR α chain as set forth in SEQ ID NO: 1 and a TCR β chain as set forth in SEQ ID NO: 2 having a S109N mutation.

According to a specific embodiment, the TCR comprises a TCR α chain as set forth in SEQ ID NO: 1 and a TCR β chain as set forth in SEQ ID NO: 2 having a T63I mutation.

According to a specific embodiment, the TCR comprises a TCR α chain as set forth in SEQ ID NO: 1 having a S189G mutation and a TCR β chain as set forth in SEQ ID NO: 2.

According to a specific embodiment, the TCR comprises a TCR α chain as set forth in SEQ ID NO: 1 having a G125A mutation and a TCR β chain as set forth in SEQ ID NO: 2.

According to a specific embodiment, the TCR comprises a TCR α chain as set forth in SEQ ID NO: 1 having a G125V mutation and a TCR β chain as set forth in SEQ ID NO: 2.

According to a specific embodiment, the TCR comprises a TCR α chain as set forth in SEQ ID NO: 1 having W55L and Y56F mutations and a TCR β chain as set forth in SEQ ID NO: 2.

According to a specific embodiment, the TCR comprises a TCR α chain as set forth in SEQ ID NO: 1 having W55L, Y56F and S189G mutations and a TCR β chain as set forth in SEQ ID NO: 2.

According to a specific embodiment, the TCR comprises a TCR α chain as set forth in SEQ ID NO: 1 having a S189G mutation and a TCR β chain as set forth in SEQ ID NO: 2 having a S109N mutation.

According to specific embodiments, the TCR has an increased avidity to the antigen as compared to a TCR having the same TCR α chain and TCR β chain amino acid sequences not comprising said at least one mutation.

Thus, according to specific embodiments, the TCR has increased avidity to MAGE-A1 peptide as set forth in SEQ ID NO: 25 as compared to a TCR comprising a TCR α chain as set forth in SEQ ID NO: 1 and a TCR β chain as set forth in SEQ ID NO: 2.

As used herein, the term “avidity” refers to a measure of the overall stability of the complex between a receptor and its ligand (e.g. TCR and its antigen). This overall stability is determined by e.g. the affinity of the receptor to the ligand, expression level, stability, clustering and flexibility of the receptor (e.g. TCR), and interaction with co-receptors (e.g. CD4 or CD8).

Methods of determining avidity, e.g. TCR avidity are well known in the art and are also described hereinabove and below, and include e.g. tetramer staining assay and activity assays.

According to specific embodiments, the increased avidity is manifested by increased affinity.

Hence, according to specific embodiments, the TCR has an increased affinity to the antigen as compared to a TCR having the same TCR α chain and TCR β chain amino acid sequences not comprising said at least one mutation.

Thus, according to specific embodiments, the TCR has increased affinity to MAGE-A1 peptide as set forth in SEQ ID NO: 25 as compared to a TCR comprising a TCR α chain as set forth in SEQ ID NO: 1 and a TCR β chain as set forth in SEQ ID NO: 2.

Methods of determining affinity are well known in the art and are also described hereinabove and below and include e.g. BiaCore, HPLC, Surface Plasmon Resonance assay (SPR) and flow cytometry (FACS).

According to specific embodiments, the TCR comprising the at least one mutation disclosed herein is capable of activating a CD3+CD3zeta+ T cell expressing same following contacting with the antigen.

According to a specific embodiments, the TCR comprising the at least one mutation disclosed herein has an increased activating capability as compared to TCR having the same TCR α chain and TCR β chain amino acid sequences not comprising said at least one mutation.

Thus, according to specific embodiments, the TCR capable of binding MAGE-A1 peptide comprising the at least one mutation disclosed herein is capable of activating a CD3+CD3zeta+ T cell expressing same following contacting with a MAGE-A1 peptide as set forth in SEQ ID NO: 25.

According to a specific embodiments, the TCR capable of binding MAGE-A1 peptide comprising the at least one mutation disclosed herein has an increased activating capability as compared to a MAGE-A1 specific TCR comprising a TCR α chain as set forth in SEQ ID NO: 1 and a TCR β chain as set forth in SEQ ID NO: 2.

Methods of determining activation of T cells are well known in the art and are also described hereinabove and below.

The TCRs of some embodiments of the invention may be synthesized and purified by any techniques that are known to those skilled in the art of peptide synthesis, such as, but not limited to, solid phase and recombinant techniques.

According to specific embodiments, production of the TCR involves solid phase synthesis.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

According to specific embodiments, the TCR is produced by recombinant DNA technology.

Thus, according to an aspect of the present invention, there is provided at least one polynucleotide encoding the TCR.

According to a specific embodiment, a single polynucleotide encodes both TCR α and β chains.

According to another specific embodiments, one polynucleotide encodes the TCR α chain and a separate polynucleotide encodes the TCR β chain.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

Non-limiting examples of polynucleotides encoding the TCR of some embodiments of the invention are provided in SEQ ID NO: 54-60.

To express any of the disclosed polypeptides in a cell, a polynucleotide sequence encoding the polypeptide is preferably ligated into a nucleic acid construct suitable for cell expression. Such a nucleic acid construct includes a promoter sequence for directing transcription of the polynucleotide sequence in the cell in a constitutive or inducible manner.

Thus, according to an aspect of the present invention there is provided a nucleic acid construct or system comprising at least one polynucleotide encoding the TCR, and a regulatory element for directing expression of said polynucleotide in a host cell.

According to specific embodiments, the promoter is heterologous to the nucleic acid sequence encoding the polypeptide.

Non-limiting Examples of promoters that can be used with specific embodiments of the invention include promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV), promoters from human genes such as human actin, human myosin, human hemoglobin, human muscle creatine and human metalothionein and tissue-specific promoters such as involucrin, keratin 5, and keratin 14.

According to specific embodiments, the promoter is an inducible promoter.

Inducible mammalian promoters are known to those of skill in the art (see, e.g. Bitter et al. (1987) Methods in Enzymology 153: 516-544). Inducible promoters can be activated by external signals or agents (i.e. inducer). The inducer may directly activate a promoter or inactivate a repressor of that promoter. For example, inducible systems endogenous to mammalian cells include promoters induced by heavy-metals (Brinster et al. Nature (1982) 296:39-42; Mayo et al. Cell (1982) 29:99-108; and Searle et al. Molecular and Cellular Biology (1985) 5:1480-1489), steroid hormones (Hynes et al. Proc. Natl. Acad. Sci. USA (1981) 78:2038-2042; Lee et al. Nature (1981) 294:228-232; and Klock et al. Nature (1987) 329:734-736), heat shock (Nouer, Heat Shock Response. Boca Raton, Fla., Ed. CRC, 1991) (reviewed in Mullick, A. and B. Massie Encyclopedia of Cell Technology pp. 1 140-1 164, 2000)) are well characterized. PCT publication WO2002/088346 discloses a cumate-inducible promoter. Additional inducible promoters are known in the art, and include, but are not limited to inflammation and hypoxia induced promoters. Prokaryotic and insect inducible promoter systems have been adapted for regulated expression in mammalian cells. See, for example, Gossen et al. (1993) TIBS 18:471-475 and No et al. (1996) Proc. Natl. Acad. Sci. USA 93:3346-3351). The insect ecdysone-inducible promoter is tightly regulated with no detectable background expression in the absence of inducer. Ecdysone is suitable for use in vivo because it is a naturally occurring lipophilic steroid that can penetrate tissues, is inert in mammals and exhibits rapid clearance kinetics (No et al). Gupta et al. (PNAS (2004) 101: 1927-1932) discloses retroviral delivery of an ecdysone-inducible gene expression system under the control of a modified RNA polymerase Ill-specific U6 promoter.

The prokaryotic repressors from the lac and tet operons have been incorporated in eukaryotic inducible expression systems. Repression of expression is mediated by the repressor bound to operator sites placed downstream of the minimal promoter in the absence of inducer and repression is relieved on the addition of the inducer (Brown et al. (1987) Cell 49:603-612; Hu and Davidson (1987) Cell 48:555-566; Blau and Rossi, Proc. Natl. Acad. Sci. USA (1999) 96:797-799; and Gossen et al. (1995) Science 268:1766-1769).

According to specific embodiments, the inducible promoter is a Tet-on promoter induced by Tetracycline or Doxycycline.

Methods for construction of nucleic acid constructs or systems containing an inducible promoter operatively linked to a coding sequence of any polypeptide are known to those of skill in the art, as are methods for introducing such constructs of systems and vectors containing such expression cassette into cells.

The nucleic acid construct or system (also referred to herein as an “expression vector”) of some embodiments of the invention includes additional sequences which render this vector suitable for replication and integration (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct or system of some embodiments of the invention typically includes or encodes a signal sequence for targeting the polypeptide to the cell surface. According to a specific embodiment, the signal sequence for this purpose is a mammalian signal sequence or the signal sequence of the TCR of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed, i.e. T cells. Examples of T cell specific promoters include lymphoid specific promoters [Calame et al., (1988) Adv. Immunol. 43:235-275]; in particular promoters of T-cell receptors [Winoto et al., (1989) EMBO J. 8:729-733].

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) or a self-cleavable peptide; and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMT010/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The ability to select suitable vectors for transforming T cells is well within the capabilities of the ordinary skilled artisan and as such no general description of selection consideration is provided herein.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Exemplary method of transducing cells with a TCR are known in the art and are disclosed e.g. in Nicholson et al. Adv Hematol. 2012; 2012:404081; Wang and Rivière Cancer Gene Ther. 2015 March; 22(2):85-94); and Lamers et al, Cancer Gene Therapy (2002) 9, 613-623.

According to specific embodiments, the expression vector is introduced into cells using electroporation.

According to specific embodiments, the expression vector is introduced into cells using viral (e.g., retroviral) infection. Introduction of nucleic acids by viral infection offers several advantages over other methods such as lipofection and electroporation, since higher transfection efficiency can be obtained due to the infectious nature of viruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for targeting the polypeptide to the desired site in a cell. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Specific embodiments of the present invention also contemplates T cells comprising the TCR described herein and method of generating and using same.

Thus, according to an aspect of the present invention, there is provided a T cell genetically engineered to expressed the TCR comprising the at least one mutation disclosed herein.

According to an additional or an alternative aspect of the present invention, there is provided a method of expressing a TCR in a T cell, the method comprising introducing into a T cell the polynucleotide encoding the TCR, under conditions which allow expression of the TCR.

Such conditions may be for example an appropriate temperature (e.g., 37° C.), atmosphere (e.g., air plus 5% CO₂), pH, light, medium, supplements and the like.

According to other specific embodiments, the introducing is effected in-vivo.

According to specific embodiments, the introducing is effected in-vitro or ex-vivo.

As used herein, the term “T cell” includes CD4+ T cells and CD8+ T cells.

According to specific embodiments, the T cell expresses an endogenous CD3 and/or CD3zeta.

According to other specific embodiments, the T cell does not express an endogenous CD3 and/or CD3zeta.

According to specific embodiments, the T cell does not expresses a flow cytometry detectable level of an endogenous CD3 and/or CD3zeta.

According to specific embodiments, the T cell expresses an exogenous CD3 and/or CD3zeta.

According to specific embodiments, the method comprising expressing in the T cell CD3 and/or CD3zeta.

According to specific embodiments, the T cell expresses an endogenous TCR.

According to specific embodiments, the T cell is expressing an endogenous TCR specific for a pathologic (diseased, e.g. cancerous) cell, i.e. recognizes an antigen presented as a peptide in the context of MHC which is overexpressed or solely expressed by a pathologic cell as compared to a non-pathologic cell.

According to other specific embodiments, the T cell does not express an endogenous TCR.

According to specific embodiments, the T cell is an effector cell.

As used herein, the term “effector T cell” refers to a T cell that activates or directs other immune cells e.g. by producing cytokines or has a cytotoxic activity e.g., CD4+, Th1/Th2, CD8+ cytotoxic T lymphocyte.

According to specific embodiments, the T cell is a CD4+ T cell.

According to other specific embodiments, the T cell is a CD8+ T cell.

According to specific embodiments, the T cell is a naïve T cell.

According to specific embodiments, the T cell is a memory T cell. Non-limiting examples of memory T cells include effector memory CD4+ T cells with a CD3+/CD4+/CD45RA−/CCR7− phenotype, central memory CD4+ T cells with a CD3+/CD4+/CD45RA−/CCR7+phenotype, effector memory CD8+ T cells with a CD3+/CD8+CD45RA−/CCR7−phenotype and central memory CD8+ T cells with a CD3+/CD8+CD45RA−/CCR7+phenotype.

According to specific embodiments, the T cells can be a primary cell, freshly isolated, stored e.g., cryopreserved (i.e. frozen) at e.g. liquid nitrogen temperature at any stage for long periods of time (e.g., months, years) for future use; and cell lines.

According to specific embodiments, the T cell is a human cell.

According to specific embodiments, the T cell is of a healthy subject.

According to specific embodiments, the T cell is of a subject suffering from a pathology (e.g. cancer).

According to specific embodiments, the T cell is a primary cell.

Methods of obtaining T cells are well known in the art. Thus, for examples, PBMCs can be isolated by drawing whole blood from a subject and collection in a container containing an anti-coagulant (e.g. heparin or citrate); and apheresis. According to other specific embodiments, the T cells are obtained from a tissue comprising cells associated with a pathology. Methods for obtaining a tissue sample from a subject are well known in the art and include e.g. biopsy, surgery or necropsy and preparing a single cell suspension thereof. Following, according to specific embodiments, the T cells are purified from the peripheral blood or from the single cell suspension. There are several methods and reagents known to those skilled in the art for purifying T cells such as leukapheresis, sedimentation, density gradient centrifugation (e.g. ficoll), centrifugal elutriation, fractionation, chemical lysis of e.g. red blood cells (e.g. by ACK), selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D.N. Weir, editor) and FLOW CYTOMETRY AND CELL SORTING (A. Radbruch, editor, Springer Verlag, 2000).

According to other specific embodiments, the T cells is a cell line. Numerous T cells lines are known and can be commercially available from e.g. ATCC. Non-limiting examples of T cell lines that can be used with specific embodiments of the present invention include BWZ.36, BW5147, Jurkat (and all Jurkat-derived lines), and T cell hybridoma 58−/−.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the T cells can be stored in a cell bank or a depository or storage facility.

Consequently, the present teachings further suggest the use of the T cells and the methods disclosed herein as, but not limited to, a source for adoptive T cells therapies.

Thus, according to an aspect of the present invention, the T cells disclosed herein are for use in adoptive cell therapy.

The T cells used according to specific embodiments of the present invention may be autologous or non-autologous; they can be syngeneic or non-syngeneic: allogeneic or xenogeneic to the subject; each possibility represents a separate embodiment of the present invention.

According to specific embodiments, the cells are autologous to the subject.

According to specific embodiments, the cells are non-autologous to the subject.

According to specific embodiments, the T cells described herein are cultured, expanded and/or activated ex-vivo prior to administration to the subject.

Methods of culturing, expanding and activating T cells are well known to the skilled in the art. For example, T cells may be activated ex-vivo in the presence of one or more molecule such as, but not limited to, an anti-CD3 antibody, an anti-CD28 antibody, anti-CD3 and anti-CD28 coated beads (such as the CD3CD28 MACSiBeads obtained from Miltenyi Biotec), IL-2, phytohemoagglutinin, an antigen-loaded antigen presenting cell [APC, e.g. dendritic cell], a peptide loaded recombinant MHC.

Since the T cells of specific embodiments of the present invention are activated upon binding of the TCR to an antigen (e.g. MAGE-A1) presented on the surface of cells, they may be used for, but not limited to, treating diseases associated cells presenting the antigen (e.g. MAGE-A1 peptide) e.g. cancer.

Thus, according to an aspect of the present invention, there is provided a method of treating a disease that can benefit from adoptive transfer of T cells in a subject in need thereof, the method comprising administering to the subject a therapeutically effective amount of T cells genetically engineered to express the TCR disclosed herein, wherein pathologic cells of said subject present a peptide identified by said TCR, thereby treating the disease in the subject.

According to an additional or an alternative aspect of the present invention, there is provided T cells genetically engineered to express the TCR disclosed herein, for use in a disease that can benefit from adoptive transfer of T cells in a subject in need thereof, wherein pathologic cells of said subject present a peptide identified by said TCR.

As used herein, the term “subject” refers to a human or non-human individual having an MHC system, such as the HLA system in humans. The subject may be of any gender and of any age.

According to specific embodiments, the subject is a human subject.

According to specific embodiments, the subject expresses HLA class I haplotype selected from the group consisting of HLA-A2, HLA-A1, HLA-A3, HLA-A24, HLA-A26, HLA-A28, HLA-A31, HLA-A33, HLA-A34, HLA-A0201, HLA-B7, HLA-B27 and HLA-B45. Other HLA class haplotypes are known and contemplated herein.

According to specific embodiments, the subject expresses an HLA-A2 haplotype.

According to specific embodiments, the subject is diagnosed with a disease (e.g., cancer) or is at risk of developing a disease (e.g., cancer).

According to specific embodiments, pathologic cells of the subject present the peptide (e.g. MAGE-A1) at a level above a predetermined threshold, as further described hereinbelow.

Thus, according to specific embodiments, the methods disclosed herein further comprise determining a level of MHC presented MAGE-A1 in a biological sample of the subject e.g. prior to administering of the T cell and treating the subject accordingly.

As used herein the term “treating” refers to inhibiting, preventing or arresting the development of a pathology (disease, disorder, or condition e.g., cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

According to specific embodiments, treatment may be evaluated by a decrease in tumor volume, a decrease in the number of tumor cells, a decrease in the number of metastases, an increase in life expectancy, or amelioration of various physiological symptoms associated with the cancerous condition.

As used herein, the phrase “a disease that can benefit from adoptive transfer of T cells” refers to a disease in which pathologic cells presenting a specific peptide drive onset and/or progression of the disease and thus adoptive transfer of T cells having a TCR that binds this peptide can have a beneficial therapeutic effect.

According to specific embodiments, pathologic cells present the peptide at a level above a predetermined threshold.

Such a predetermined threshold can be experimentally determined by comparing presentation levels in a biological sample derived from subjects diagnosed with the disease (e.g. caner) to a biological sample obtained from healthy subjects (e.g., not having the disease e.g. cancer). Alternatively or additionally, such a predetermined threshold can be experimentally determined by comparing presentation levels in pathologic cells (e.g. cancer cells) to presentation levels in healthy cells obtained from the same subject. Alternatively, such a level can be obtained from the scientific literature and from databases.

According to specific embodiments, the level above a predetermined threshold is statistically significant.

According to specific embodiments the increase from a predetermined threshold is at least 5%, at least 10%, at least 20%, at least 30%, at least 40%, at least 50%, at least 60%, at least 70%, at least 80%, at least 90%, at least 95%, at least 100% or more, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more as compared to the control sample as measured using the same assay.

Methods of determining presentation of the peptides are known in the art, and include e.g. flow cytometry, immunohistochemistry and the like, which may be effected using e.g. antibodies specific to the peptide.

According to specific embodiments, the disease can benefit from modulating immune cells.

As used herein the phrase “a disease that can benefit from modulating immune cells” refers to diseases in which the subject's immune response activity may be sufficient to at least ameliorate symptoms of the disease or delay onset of symptoms, however for any reason the activity of the subject's immune response in doing so is less than optimal.

According to specific embodiments, the disease can benefit from activating immune cells.

Non-limiting examples of diseases that can benefit from activating immune cells include hyper-proliferative diseases, diseases associated with immune suppression, immunosuppression caused by medication (e.g. mTOR inhibitors, calcineurin inhibitor, steroids) and infections.

According to specific embodiments, the disease comprises an infection.

As used herein, the term “infection” or “infectious disease” refers to a disease induced by a pathogen. Specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to specific embodiments of the present invention include, but are not limited to, human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the disease comprises a hyper-proliferative disease.

According to specific embodiments, the hyper-proliferative disease comprises sclerosis, fibrosis, Idiopathic pulmonary fibrosis, psoriasis, systemic sclerosis/scleroderma, primary biliary cholangitis, primary sclerosing cholangitis, liver fibrosis, prevention of radiation-induced pulmonary fibrosis, myelofibrosis or retroperitoneal fibrosis.

According to other specific embodiments, the hyper-proliferative disease comprises cancer.

Thus, according to specific embodiments the pathological cell is a cancerous cell.

According to specific embodiments, the cancer presents a MAGE-A1 peptide as set forth in SEQ ID NO: 25.

Hence, according to an aspect of the present invention, there is provided a method of treating cancer presenting a MAGE-A1 peptide as set forth in SEQ ID NO: 25 in a subject in need thereof, the method comprising administering to the subject a therapeutically effective amount of a T cells genetically engineered to express the TCR disclosed herein, thereby treating the cancer in the subject.

According to an additional or an alternative aspect of the present invention, there is provided T cells genetically engineered to express the TCR disclosed herein, for use in treating cancer presenting a MAGE-A1 peptide as set forth in SEQ ID NO: 25 in a subject in need thereof.

Cancers which may be treated by some embodiments of the invention can be any solid or non-solid tumor, cancer metastasis and/or a pre-cancer.

According to specific embodiments, the cancer is a malignant cancer.

Examples of cancer include but are not limited to, carcinoma, blastoma, sarcoma and lymphoma. More particular examples of such cancers include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcino sarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the cancer is a pre-malignant cancer.

Pre-cancers are well characterized and known in the art (refer, for example, to Berman J J. and Henson D E., 2003. Classifying the pre-cancers: a metadata approach. BMC Med Inform Decis Mak. 3:8). Examples of pre-cancers include, but are not limited to, acquired small pre-cancers, acquired large lesions with nuclear atypia, precursor lesions occurring with inherited hyperplastic syndromes that progress to cancer, and acquired diffuse hyperplasias and diffuse metaplasias. Non-limiting examples of small pre-cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia).

Non-limiting examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Non-limiting examples of precursor lesions occurring with inherited hyperplastic syndromes that progress to cancer include atypical mole syndrome, C cell adenomatosis and MEA. Non-limiting examples of acquired diffuse hyperplasias and diffuse metaplasias include Paget's disease of bone and ulcerative colitis.

According to specific embodiments, the cancer is selected from the group consisting of multiple myeloma, melanoma, neuroblastoma, liver, lung, breast, colon, bladder, stomach, thyroid, kidney, skin, and ovarian cancer.

According to specific embodiments, the disease can benefit from inhibiting immune cells.

According to specific embodiments, the disease is an autoimmune disease. Such autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to specific embodiments, the disease is graft rejection disease.

Examples of diseases associated with transplantation of a graft include, but are not limited to, graft rejection, chronic graft rejection, subacute graft rejection, hyperacute graft rejection, acute graft rejection and graft versus host disease.

According to specific embodiments, the disease is an allergic disease.

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

The T cells disclosed herein can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the T cells comprising the TCR disclosed herein accountable for the biological effect.

Thus, according to specific embodiments, the T cells are the only active ingredient in the formulation.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, intradermal, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

According to a specific embodiment, the immune cells of the invention or the pharmaceutical composition comprising same is administered via an IV route.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Alternative embodiments include depots providing sustained release or prolonged duration of activity of the active ingredient in the subject, as are well known in the art.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro, cell culture assays and animal models. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to specific embodiments, the T cells comprising the TCR disclosed herein can be administered to a subject with other established or experimental therapeutic regimen to treat a disease associated with cells presenting a peptide (e.g. cancer) including analgetics, chemotherapy, radiotherapy, phototherapy and photodynamic therapy, surgery, nutritional therapy, ablative therapy, combined radiotherapy and chemotherapy, brachiotherapy, proton beam therapy, immunotherapy, antibodies, cellular therapy, photon beam radiosurgical therapy and other treatment regimens which are well known in the art.

According to an aspect of the present invention there is provided an article of manufacture comprising the T cells comprising the TCR disclosed herein and an additional therapy for the disease (e.g. cancer therapy).

According to specific embodiment, the T cells comprising the TCR disclosed herein and the additional therapy for the disease are packaged in separate containers.

According to specific embodiment, the T cells comprising the TCR disclosed herein and the additional therapy for the disease are packaged in a co-formulation.

According to specific embodiments, the article of manufacture is identified for the treatment of the disease (e.g. cancer).

As the present inventors have developed a novel method of inducing mutations in a TCR using SHM, specific embodiments of the present invention contemplate using this methodology in inducing mutations in a TCR in order to modulate its avidity and thereby activity.

Hence, according to an aspect of the present invention, there is provided a method for modulating the avidity of a T cell receptor (TCR) to its ligand, the method comprising:

(a) expressing in a T cell a nucleic acid sequence encoding the TCR, the nucleic acid sequence has been codon optimized to:
(i) maximize the number of WRCH (SEQ ID NO: 5) or DGYW (SEQ ID NO: 6) nucleic acid sequences,
(ii) minimize the number of SYC or GRS nucleic acid sequences,
(iii) maximize the number of WA nucleic acid sequences and/or
(iv) minimize the number of rare codons; and

(b) expressing in the T cell Activation Induced cytidine Deaminase (AID) having an amino acid sequence as set forth in SEQ ID NO: 7.

Specific embodiments of the present invention contemplate using SHM in inducing mutations in a Chimeric antigen receptor (CAR) in order to modulate its avidity and thereby activity.

Hence, according to an aspect of the present invention, there is provided a method for modulating the avidity of a or a Chimeric antigen receptor (CAR) to its ligand, the method comprising:

(a) expressing in a cell a nucleic acid sequence encoding the CAR; and

(b) expressing in the cell Activation Induced cytidine Deaminase (AID).

According to specific embodiments, the nucleic acid sequence expressed in the (a) has been codon optimized to:
(i) maximize the number of WRCH (SEQ ID NO: 5) or DGYW (SEQ ID NO: 6) nucleic acid sequences,
(ii) minimize the number of SYC or GRS nucleic acid sequences,
(iii) maximize the number of WA nucleic acid sequences and/or
(iv) minimize the number of rare codons.

According to specific embodiments, the nucleic acid sequence has been codon optimized to at least (i), at least (ii), at least (iii), at least (i)+(ii) or at least (i)+(ii)+(iii).

According to specific embodiments, the nucleic acid sequence expressed in said (a) has been codon optimized to include a CAGGTG (SEQ ID NO: 27) sequence.

As used herein the phrase “chimeric antigen receptor (CAR)” refers to a recombinant or synthetic molecule which combines an extracellular antibody-based domain specific for a desired antigen with a T cell receptor-activating intracellular domain to generate a chimeric protein that exhibits cellular immune activity to the specific antigen.

Non-limiting examples of cells that can be used with specific embodiments of the present invention include T cells, Chinese Hamster Ovary (CHO), HEK293, NIH-3T3, PER.C6, HT1080, NS0, Sp2/0, BHK, Namalwa, COS, HeLa and Vero cell.

According to specific embodiments, the cell is a T cell.

Method of expressing a nucleic acid sequence in a cells are known in the art and are further described in details hereinabove and in the Examples section which follows.

Specific non-limiting examples of expressing a nucleic acid sequence encoding a CAR are disclosed e.g. in Davila et al. Oncoimmunology. 2012 Dec. 1; 1(9):1577-1583; Wang and Rivière Cancer Gene Ther. 2015 March; 22(2):85-94); and Maus et al. Blood. 2014 Apr. 24; 123(17):2625-35.

The nucleic acid sequences encoding the TCR or CAR of some embodiments of the invention are codon optimized to optimize SHM process.

Therefore, a codon optimized nucleic acid sequence refers to a sequence in which the nucleotide sequence of a native or naturally occurring sequence has been modified without affecting the encoded amino acid residue (due to the degeneracy of the genetic code) in order to utilize statistically-preferred or statistically-favored codons which enable SHM.

According to specific embodiments, the codon optimization is effected at the region interacting with the antigen e.g. CDR3 for TCR, CDR1/2/3 for CAR.

As used herein, the phrase “maximize the number of nucleic acid sequences” refers to at least 5 repeats of the recited nucleic acid sequence.

As used herein, the phrase “minimize the number of nucleic acid sequences” refers to no more than 5 repeats of the recited nucleic acid sequence.

Rare codons are codons that are less than half as frequent as the most frequently used codon for a specific amino acid according to a human codon usage table.

As used herein, the phrase “minimize the number of rare codons” refers to no more than 5 rare codons.

According to specific embodiments, following the codon optimization step the selected nucleic acid sequence expressed is the one having the highest codon adaptation index (CAI).

The codon adaptation index (CAI) is a known method based on the codon usage of highly expressed genes, as described e.g. in “The Codon Adaptation Index—a measure of directional synonymous codon usage bias, and its potential applications” by Sharp & Li 1987, the contents of which are fully incorporated herein by reference. The calculation yields a score of how similar the codon usage of the sequence in question is to the codon usage in that gene.

As used herein the phrase “modulating affinity” refers to a change in affinity of a TCR or a CAR to its ligand following introduction of mutations in its sequence as compared to the affinity of the reference TCR or CAR to the same ligand (i.e., prior to subjecting the method disclosed herein). The change can be an increase or a decrease. Methods of determining affinity are well known in the art and are further described in details hereinabove and below.

To introduce such mutations the method comprises expressing in the cell (e.g. T cell) Activation Induced cytidine Deaminase (AID).

Activation Induced cytidine Deaminase (AID) is an enzyme classified into the APOBEC family of polynucleotide Cytidine deaminases, which perform hydrolytic deamination of Cytidine (C) to Uridine (U).

According to specific embodiments the AID is human AID.

According to specific embodiments the AID is human AID, such as provided in e.g. NP_001317272, NP_065712.

The AID of some embodiments of the invention also encompasses functional homologues (naturally occurring or synthetically/recombinantly produced), which exhibit the desired activity [i.e., perform hydrolytic deamination of Cytidine (C) to Uridine (U)].

According to specific embodiments, the amino acid sequence of AID may comprise conservative and/or non-conservative amino acid substitutions. Detailed description on conservative and non-conservative amino acid substitutions is provided hereinabove.

The AID used with specific embodiments of the present invention is an active variant of human AID, known as AID mut 7.3, having an amino acid sequence as set forth in SEQ ID NO: 7.

According to specific embodiments, expressing the AID comprises transiently expressing AID. Methods of transient expression are well known in the art and include, but not limited to, expression under the control of an inducible promoter (e.g. Tet-on promoter, as further described in details hereinabove and in the Examples section which follows) or by introduction of AID mRNA into the cells (in this way the mRNA will be translated in the cells and degraded after a relatively short time).

According to specific embodiments, the ligand comprises a TAA.

According to specific embodiments, the ligand is a MAGE-A1 peptide as set forth in SEQ ID NO: 25.

The change in avidity can be of at least 5%, 10%, 30%, 40% or even higher say, at least 50%, 60%, 70%, 80%, 90% or more than 99% as compared to the avidity of the reference TCR or CAR to the same ligand as determined by e.g. tetramer staining. According to specific embodiments, the change is at least 1.5 fold, at least 2 fold, at least 3 fold, at least 5 fold, at least 10 fold, or at least 20 fold as compared to the avidity of the reference TCR or CAR to the same ligand as determined by e.g. tetramer staining.

Thus, according to specific embodiments, the method further comprising selecting cells expressing a TCR or a CAR with increased or decreased avidity to the ligand as compared to the TCR or CAR prior to expression of the AID.

According to specific embodiments, the selecting is effected by tetramer staining followed by high-throughput sequencing.

According to specific embodiments, the obtained TCR or CAR is further qualified by determining activation following contacting with the ligand. Methods of determining activation, such as, but not limited to cytokine production, expression of surface marker, in-vitro and in-vivo cytotoxic assays, are well known in the art and are further described in details hereinabove and below.

According to specific embodiments, the method further comprising selecting cells expressing a TCR or a CAR with increased or decreased activity following contacting with said ligand as compared to said TCR or said CAR prior to said expressing.

According to specific embodiments, the expressing and the selecting steps are effected at least twice or at least three times.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

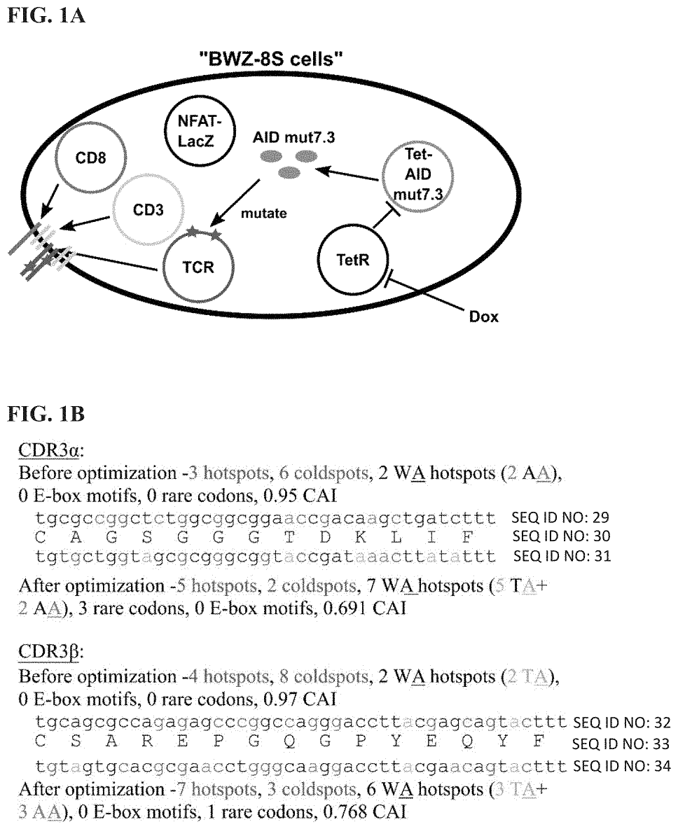

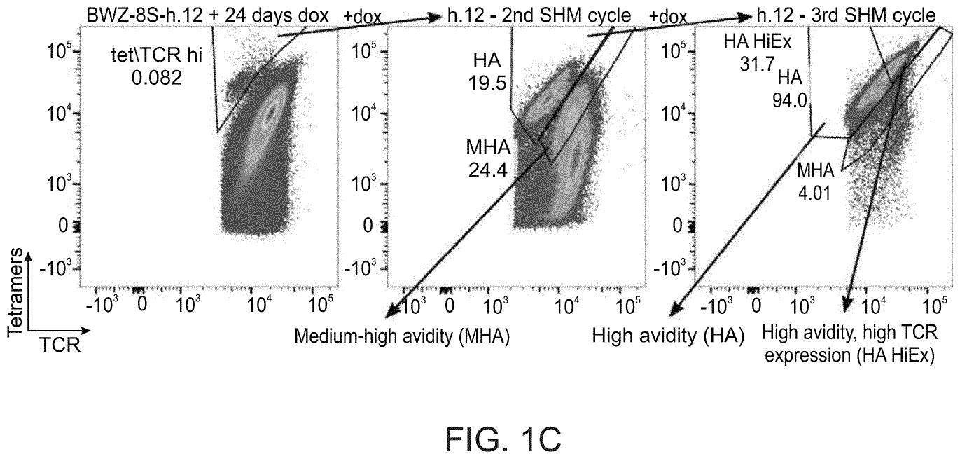

Optimization of DNA sequences for somatic hypermutation (SHM)—The DNA sequences encoding the TCR alpha and TCR beta chains of MAGE-A1 specific TCR, hT27 (SEQ ID NO: 3 and 4, respectively), were optimized at their CDR3 domains as follows (see FIG. 1B): AID hot-spots [WRCH (SEQ ID NO: 5)/DGYW (SEQ ID NO: 6)] were maximized; AID cold-spots, SYC/GRS were minimized; Pol η hotspots [WA, with preference for TA_(32, 33)] were maximized; tandem rare codons were minimized and the codon adaptation index (CAI) was maximized.

Plasmids and cloning—Cloning of the pBABE-CD3 and pcDNA4-Tet-hAID was effected as described in (14). The pCL-Ampho vector was as described in Naviaux et al. (1996). The pCL-Eco and codon-optimized pMSGV1-Pmel-1 TCR vector (codon optimized Pmel-1α and β chains separated by a T2A segment) were as described in (24). The codon-optimized MP71-hT27 TCR and MP71-T1367 TCR vectors containing mouse constant regions were as described in (10). The mutant MP71-hT27 TCR vectors and pcDNA4-Tet-hAID mut 7.3 vector (25), were generated using site directed mutagenesis (SDM) with the Phusion SDM Kit (Thermo-Fischer Scientific, Waltham, Mass., USA) according to the manufacturer's protocol. The pCDNA6-TR vector, containing the Tet-repressor (TetR or TR), was obtained from Invitrogen (Carlsbad, Calif., USA, Cat no: V102520). The pMSGV1-TetR vector was generated using restriction-free (RF) cloning, as previously described (26), with the Phusion HSII HF polymerase (Thermo-Fischer Scientific) according to the manufacturer's protocol. The following primers were used for sequencing:

### MP71 Forward: ATTTGTCTGAAAATTAGCTCGA (SEQ ID NO: 9),

### MP71 Reverse: AGAGCAACTACAGCTACTGC (SEQ ID NO: 10),

hT27 internal Forward: CATTTAAATGTATACCCAAATCAA (SEQ ID NO: 11), pMSGV1

### Forward: CCTCAAAGTAGACGGCATCG (SEQ ID NO: 12, Sigma-Aldrich, Rehovot, Israel).

Cell lines—Phoenix-ampho (ATCC, CRL-3213) and Platinum-Eco (Plat-E) cells (Cell Biolabs, RV-101). were cultured in “cDMEM” containing DMEM (GibcoBRL, Grand Island, N.Y., USA) supplemented with 10% FCS (GibcoBRL), 200 mM L-Glutamine, 100 mM sodium pyruvate, 1×non-essential amino acids, and 50 μg/ml Gentamicin (all Biological Industries, Beit Ha-emek, Israel). 721.211-A2 [LCL-721.221 cells transfected with HLA-A2, as they do not naturally express HLA-I molecules (27)], BWZ.36 (23), DLD1(ATCC, CCL-221), EL4-HHD [EL4 cells from a mouse lymphoma that were stably transfected with an β₂m-HLA-A2-D^{b} single chain molecule (HHD) (28)], and T2 cells (ATCC, CRL-1992), were cultured in “cRPMI” containing RPMI 1640 (GibcoBRL) supplemented with 10% FCS, 200 mM L-Glutamine, 100 mM sodium pyruvate, lx non-essential amino acids, 50 μg/ml Gentamicin, and 50 μM β-mercaptoethanol. Cell lines were all checked for mycoplasma using the EZ-PCR Mycoplasma Test Kit (Biological Industries), and were all found to be negative. Cell lines were generally used between one week and one month after thawing. Human PMBCs were isolated from leukocyte samples from the Magen David Adom blood bank (Ramat Gan, Israel) using Ficoll-Paque (GE Healthcare, Chicago, Ill., USA) and frozen in aliquots of 2×10⁷ cells/vial or cultured in cRPMI supplemented with 300 U/ml rh-IL-2 (ProSpec, Ness Tziona, Israel).

Flow cytometry and fluorescence-activated cell sorting (FACS)—For flow cytometry cells were stained with antibodies for 30 minutes at 4° C. using the dilution recommended by the manufacturer, unless indicated otherwise. Anti-mouse APC-CD3 and anti-human APC-IFNγ, FITC-CD8 (1:100), PE-Cy7-IL2 (1:50), and PerCP-eFlour710-CD4 (1:100) antibodies were purchased from eBioscience (San Jose, Calif., USA), and anti-mouse PE-TCRβ (1:333) from BioLegend (San Diego, Calif., USA). Viability staining was performed with either Zombie-aqua (1:500, BioLegend) for cells to be fixed and permeabilized, or 1 μM DAPI (BioLegend) for other cells. APC-conjugated HLA-A2-MAGE-A1₂₇₈₋₂₈₆ were prepared by combining biotinylated monomers from the NIH tetramer facility (Bethesda, Md., USA) and APC-Streptavadin (eBioscience) mixed at a 4:1 molar ratio. APC-Streptavadin was added to the monomers in 5 portions separated by 20 minutes. Cells were stained with tetramers for 1 hour at 4° C. using a 1:200 dilution unless otherwise noted, and then stained with antibodies. BWZ-derived cells were sorted via FACS using a 100 μm nozzle, with 100 nM DAPI added immediately before running. All results were analyzed using FlowJo software (ThreeStar, San Carlos, Calif., USA).

Electroporations—BWZ.36-derived cells were electroporated with 5 μg of linearized DNA for 2 ms at 400V with the ECM 830 electroporator (BTX, Hollistone, Mass., USA) at a density of 2×10⁷ cells/ml in Opti-MEM (GibcoBRL), 250 μl (5×10⁶ cells) per 4 mm cuvette. Selection of cells with stable expression of TetR (on the pCDNA6-TR vector) was performed for 2 weeks using 6 μg/ml blastidin (Invitrogen). Subsequently, selection of cells with stable expression of AID or AID mut 7.3 (on the pCDNA4 vector) was performed for 2 weeks using 600 mg/ml zeocin (Invitrogen). Cells were maintained in 3 μg/ml blastidin and 300 mg/ml zeocin. Following SHM, selection antibiotics were not added to the medium.

RNA extraction and reverse transcriptase (RT)-PCR—RNA was extracted from cells using the RNeasy Mini kit (Qiagen, Hilden, Germany). RT-PCR to generate cDNA from mRNA was performed with the Tetro RT-PCR kit (Bioline, London, UK) using oligo-dT primers. Gene expression from cDNA was performed using PCR with the following gene-specific primers:

AID Forward:
(SEQ ID NO: 13)
ATGGACAGCCTCTTGATG,
AID Reverse:
(SEQ ID NO: 14)
TCAAAGTCCCAAAGTACG,
TetR Forward:
(SEQ ID NO: 15)
CGTAAACTCGCCCAGAAG,
TetR Reverse:
(SEQ ID NO: 16)
AGTAAAATGCCCCACAGCG,
mGAPDH Forward:
(SEQ ID NO: 17)
CGTGTTCCTACCCCCAATGT,
mGAPDH Reverse:
(SEQ ID NO: 18)
TGTCATCATACTTGGCAGGTTTCT,
MAGE-A1 Forward:
(SEQ ID NO: 19)
CAACTTCACTCGACAGAGGCA,
MAGE-A1 Reverse:
(SEQ ID NO: 20)
CCTAGGCAGGTGACAAGGAC,
hGAPDH Forward:
(SEQ ID NO: 21)
TCACCAGGGCTGCTTTTAACT,
hGAPDH Reverse:
(SEQ ID NO: 22)
GCCATGGGTGGAATCATATTGG.
All primers were
synthesized by Sigma-Aldrich.

Retroviral transductions—Retroviruses were produced in Phoenix-ampho cells. Cells were seeded on 6-well plates, 8×10⁵ cells/well, grown to 70-90% confluence, and transfected with 1.5 μg of the target vector and 0.5 μg pCL-Ampho (gag/pol/ampho-env) using Lipofectamine2000 (Invitrogen). The supernatant containing viruses was harvested 42 hours post-transfection and cell debris was removed with 0.45 μm filters.

PMBCs were thawed, washed, and suspended in cRPMI supplemented with 50 ng/ml anti-human CD3c (eBioscience, clone OKT-3) and 300 U/ml rh-IL2. Cells were seeded at a density of 2×10⁶ cells/ml, 1 ml per well of a 24-wells plate, and incubated for 40 hours at 37° C. Non-tissue culture plates were coated with retronectin (Takara Bio, Otsu, Japan), viruses were added (2 ml per well), plates were centrifuged at 2000 g for 2 hours at 32° C. without brakes, and 1.5 ml of supernatant was removed. Activated PMBCs were added, 1×10⁶ cells in 1.5 ml per well, and centrifuged at 1500 rpm for 10 minutes without brakes.

BWZ.36-derived cells were transduced in 24-well plates by mixing 4×10⁵ cells in 200 μl with 1 ml of viruses (undiluted or with the indicated dilution of viruses) in the presence of 4 μg/ml protamine sulfate (Sigma-Aldrich). Plates were centrifuged at 1000 g for 1.5 hours at 32° C. without brakes and incubated overnight. Selection of cells with stable expression of CD3 (on the pBABE-CD3 vector) was performed for 2 weeks using 0.5 μg/ml puromycin (Invitrogen).

SHM and sorting cycles to select avidity-enhanced hT27 TCRs—SHM-ready BWZ-derived cells were transduced with the hT27 TCR and sorted, one cell per well. SHM was initiated by adding doxycline (“dox”, Sigma-Aldrich), an analog of tetracycline, at a concentration of 1 μg/ml following expansion and selection of clones based on mTCRβ expression. After 24 days cells with an increased tetramer/TCR staining ratio were sorted. Additional SHM cycles were effected 10-14 days of incubation with dox followed by sorting, 5000 cells per well. Following two or three total cycles cells were sorted into a number of groups for sequencing and avidity analysis.

High throughput SMRT sequencing with PacBio Sequel system—Genomic DNA (gDNA) was extracted using the DNeasy Blood & Tissue kit (Qiagen). hT27 TCR was amplified from gDNA using the Phusion Hotstart II HF polymerase. Primers containing a tag (underlined), 8N unique molecular identifiers (UMIs), and an MP71 specific sequence (uppercase) were used for the first two PCR cycles to add UMIs:

Forward:
(SEQ ID NO: 23)
gactgtacagtgatcgtacgnnnnnnnn
TCCAAGCTCACTTACAGGCGG,
and
Reverse:
(SEQ ID NO: 24)
ctgatcgatcgtcaactagcnnnnnnnn
TGGCGGTAAGATGCTCGAATTC
(Sigma-Aldrich).

Following, the product was purified and amplified with primers of the underlined tag for 35 PCR cycles. The final product was purified with PacBio AMPure beads, library prepared with the SMRTbell barcoded adapter kit, and samples run on a PacBio Sequel System (Pacific Biosciences, Menlo Park, Calif., USA). For analysis, demultiplexing followed by circular consensus sequence (CCS) analysis was performed using SMRT Link v5.0, and further analysis in UNIX using bwa and samtools, and mutations were visualized using the Integrated Genomics Viewer (IGV).

Peptide-loading of T2 cells for in-vitro functional assays—T2 cells were pulsed with the indicated peptides at the indicated concentrations for 2 h in Opti-MEM. MAGE-A1₂₇₈₋₂₈₆ [KVLEYVIKV (SEQ ID NO: 25)] and MUC1₁₃₋₂₁ [LLLTVLTVV (SEQ ID NO: 26)] peptides with >99% purity were synthesized by Sigma-Aldrich. Crude peptides for MAGE-A1₂₇₈₋₂₈₆ [KVLEYVIKV (SEQ ID NO: 25)], alanine substitution library, and potential cross-reactive peptides were synthesized by Genemed Synthesis (San Antonio, Tex., USA).

TCR activation assay in BWZ.36-derived cells detected with CPRG—Transduced BWZ.36-derived cells, 6×10⁴ per well, were co-cultured with target cells, 4×10⁴ per well, for 6 hours at an E:T (Effector:Target) ratio of 1.5:1. Plates were centrifuged at 2300 RPM for 2 minutes, washed with 100 μl PBS+/+ and lysed in a solution containing 1.75 mg chlorophenolred-ß-D-galactopyranoside (CPRG, Sigma-Aldrich), 90 μl 1M MgCl2, and 12.5 μl NP40 in 10 ml PBS−/− per plate. Optical density (O.D.) of 570 nm, with a reference of 630 nm, was monitored and the 40.D was calculated.

In-vitro cytokine production assay—Production of IFNγ and IL2 was detected by intracellular staining. Transduced PBMCs, 1.5×10⁵ per well, were co-cultured with target cells, 1×10⁵ per well, for 6 hours at an E:T (Effector:Target) ratio of 1.5:1. Brefeldin A (“BFA,” eBioscience) was added for the last 4 hours of the co-culture for prevention of cytokine secretion. Following, cells were stained for viability with Zombie-aqua, fixed, permeabilized, and stained for hCD4, hCD8, hIL2, hIFNγ, and mTCRβ and analyzed by flow cytometry. Assays were performed in duplicates.

In-vitro cytotoxicity assay—In-vitro cytotoxicity was evaluated by a S³⁵-methionine release assay. Target cells were labelled with S³⁵-methionine (PerkinElmer, Waltham, Mass., USA) overnight. Following, transduced PBMCs were co-cultured with the labelled target cells, 5×10³ per well, for 5 hours at the indicated E:T ratios. Target cells alone were used for determining spontaneous release, and 50 mM NaOH was added for determining total release. Plates were then centrifuged, 50 μl of the supernatant was transferred to a new plate, and 150 μl MicroScint 40 (PerkinElmer) was added to each well. S³⁵-methionine released into the medium due to specific tumor lysis was detected with a Micro β counter (PerkinElmer). Percent specific lysis was calculated as 100×(sample release−spontaneous release)/(total release—spontaneous release). If sample release was lower than the spontaneous release, it was considered as no killing. Assays were performed in triplicates.

Determining presence of motifs in proteins—The FuzzPro algorithm was used to search the human and mouse Uniprot database for the motif xxLEYxxxx (SEQ ID NO: 35). Peptides containing the motif were analyzed for predicted binding to HLA-A*02:01 using the IEDB MHC-I binding algorithm. Peptides with a predicted IC₅₀ less than 255 nM are predicted to bind.

Structural modeling—The structural model for the variable regions of the hT27 TCR was built using TCRmodel, as previously described (29). The orientation of the TCR chains and MHC was derived from the AGA1 TCR (PDB ID: 2YPL) structure, which contains HLA-B*5703 MHC, KF11 peptide from HIV, and the AGA1 TCR. The structural model for the murine constant regions of hT27 TCR was taken from the structure of the mouse 2C TCR (PDB ID: 1TCR). Mutations were simulated using Swiss-PDB Viewer (Swiss Institute of Bioinformatics, Lausanne, Switzerland).

Statistical analysis—Statistical analysis was performed using GraphPad Prism 6.0. All multiple comparisons following ANOVA used Tukey's correction for honest significant difference. EC₅₀ values were calculated on log 10-transformed values with non-linear regression of log (agonist) vs. response (three parameters). All p-values are two-sided and following Tukey's correction.

### Example 1

### Generation of High Avidity Mage-A1 Specific TCRs

The present inventors sought to use somatic hypermutation (SHM) to enhance avidity of the MAGE-A1 specific TCR, hT27. To this end, the T cell line, BWZ.36-CD8a (23, 30) was transduced with a polynucleotide encoding the alpha and beta chains of hT27 (SEQ ID NO: 1 and 2, respectively). BWZ.36-CD8a do not express an endogenous TCR and carry an NFAT-LacZ reporter gene thus express β-galactosidase (β-gal) upon TCR activation. This allows for expression of a TCR of choice and detection of TCR activation by the color change of chlorophenolred-ß-D-galactopyranoside (CPRG) from yellow to red due to cleavage by β-gal. To optimize SHM, the DNA sequence encoding the hT27 TCR alpha and beta chains were codon optimized (see FIG. 1B). In nature, the DNA sequence of antibodies, which undergo SHM in B cells, has been fine tuned for SHM in the complementary determining regions (CDRs) that interact with the antigen (31). To this end, an algorithm was developed to mimic this process for the DNA sequence of TCRs in the CDR3 loops, which interacts with the peptide. Specifically, AID hotspots, WRCH (SEQ ID NO: 5)/DGYW (SEQ ID NO: 6), were maximized and AID coldspots, SYC/GRS were minimized. Further, as mutations initiated by AID can recruit error prone DNA machinery which can lead to additional mutations, including Pol η(32, 33); Pol η hotspots, WA, with preference for TA (32, 33) were maximized. When possible, the E-box motif CAGGTG, important in E47-mediated recruitment of AID (34, 35) was included. Lastly, to maintain high expression the number of tandem rare codons was minimized and the codon adaptation index (CAI) (36) was maximized.

Furthermore, the BWZ.36-CD8α cells were transduced with a polynucleotide encoding CD3 (SEQ ID NO: 28) to compensate for low endogenous expression. In addition, the BWZ.36-CD8α cells were engineered to express an active variant of human AID, known as AID mut 7.3 (25) (SEQ ID NO: 7) under a tetracycline-inducible promoter (Tet-AIDmut7.3). The cells were also transduced with TetR to ensure that AID expression is dox-dependent (FIG. 8).

These genetically engineered BWZ.36-CD8a are referred to herein as “BWZ-8S” (FIG. 1A).

To ensure that cells contain only one TCR copy, a very low multiplicity of infection (MOI) was used, resulting in about 1% TCR+ cells. The positive cells were sorted, and SHM was induced by adding dox to four clones (h.5, 7, 8 and 12).

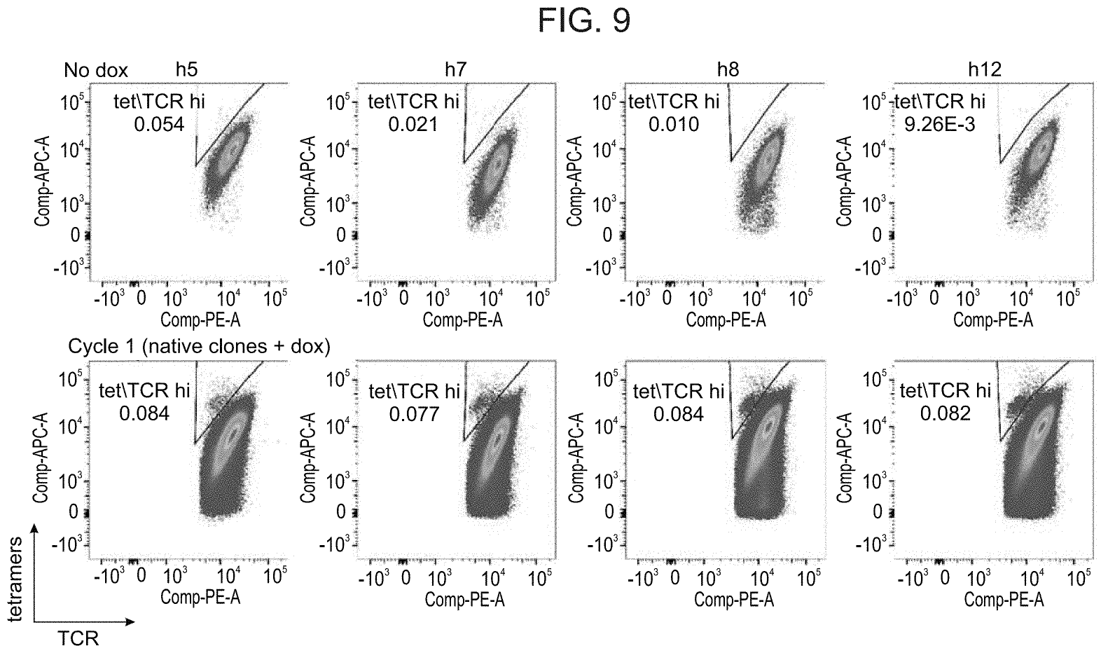

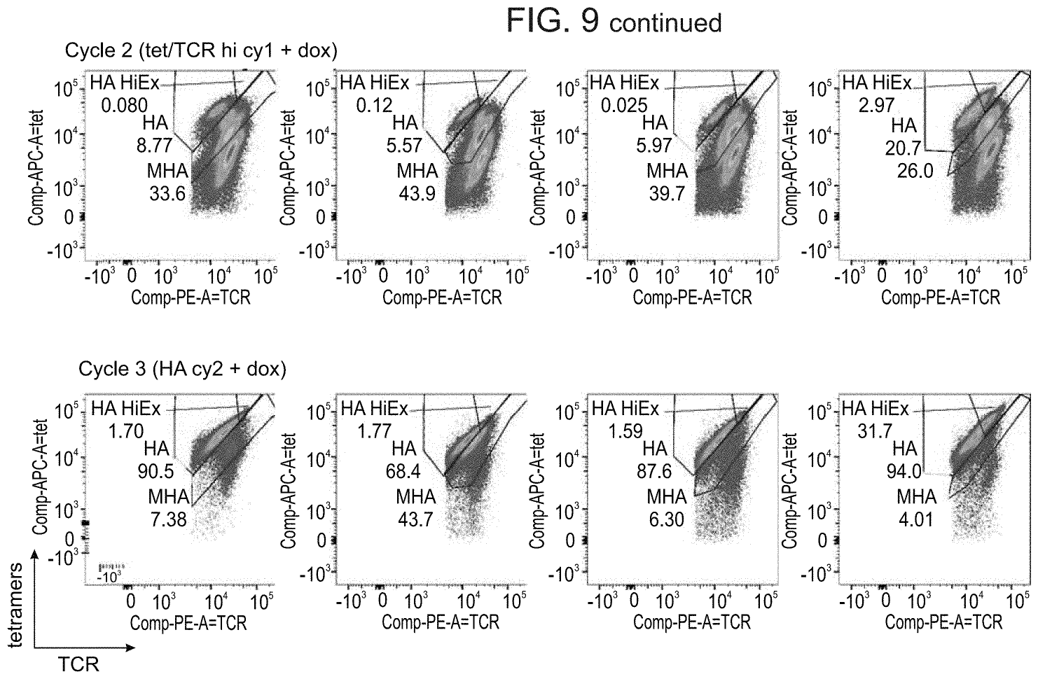

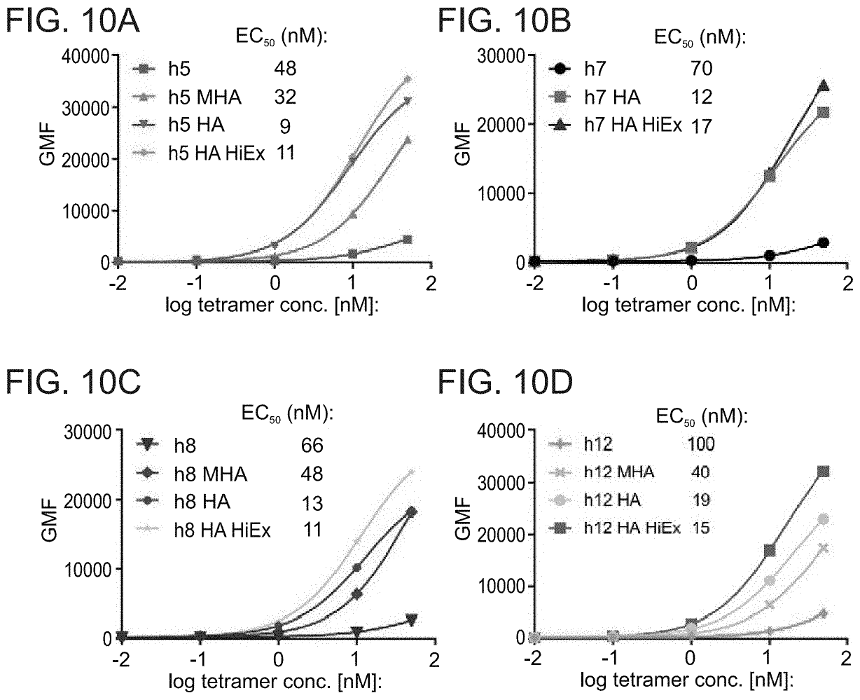

Following 24 days, cells with an increased tetramer binding at a given TCR expression level, indicative of enhanced avidity, were sorted. The sorted cells underwent a second SHM and sorting cycle in which there were 3 distinct populations relative to the native low-avidity TCR: no change in avidity, medium-high avidity (“MHA”), and high-avidity (“HA”). The HA population underwent a third SHM and sorting cycle. No further shift in tetramer/TCR ratio was observed, but there were some high-avidity cells with high TCR expression (“HA HiEx”). 5000 cells from each group, namely MHA from the second cycle and HA (including HA HiEx) from the third cycle (FIGS. 1C and 9) were sorted. Sorted groups had considerably enhanced tetramer binding than the parental lines (FIGS. 10A-D).

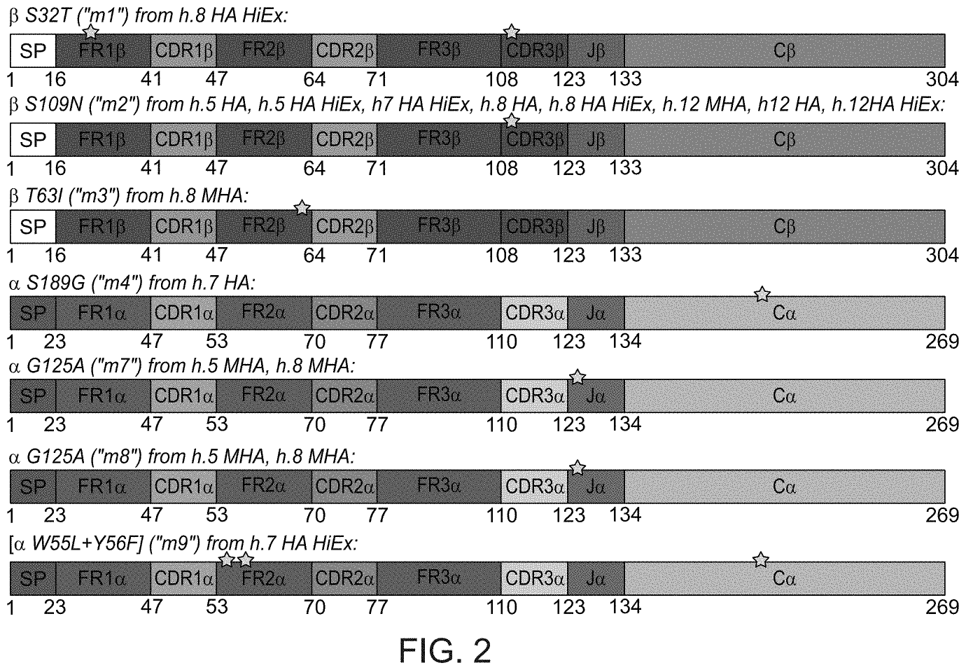

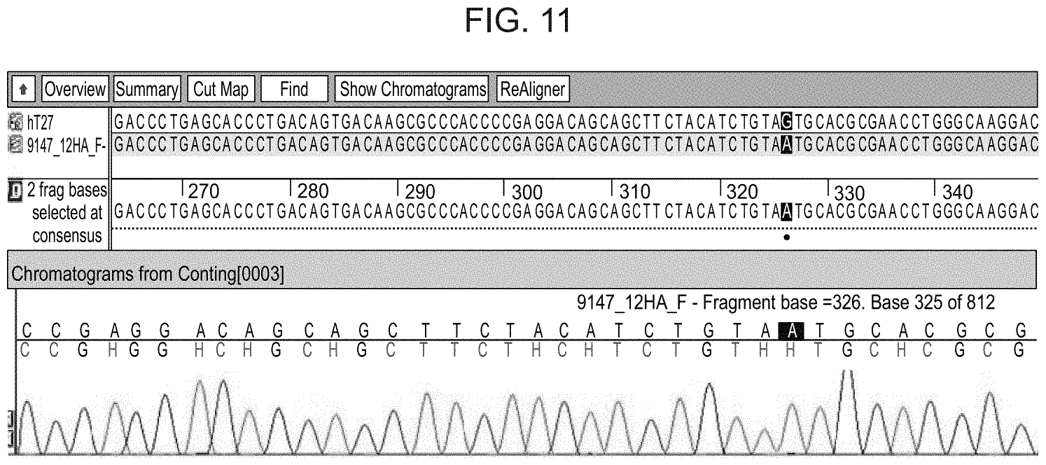

To identify the mutations responsible for the shift in tetramer binding, single-molecule real-time (SMRT) sequencing with Sequel platform (37) was used. This technology generates long-reads containing the entire 2 kb TCR sequence, allowing detection of mutations in distant regions on the same TCR. The sequencing results were demultiplexed and circular consensus sequences (CCS) were built from reads with >7 passes of the 2 kb sequence with a predicted accuracy above 99.9%. 11 unique DNA mutations were identified, represented alone or in combination, in eight mutant TCRs. Overall, 8 unique missense mutations were detected (FIG. 2 and tables 1A-B hereinbelow). For example, Sanger sequencing suggests that nearly all cells in the h.12 HA sample, contain the G326A mutation on the DNA sequence of the TCR beta chain, which leads to a S109N replacement (FIG. 11). Notable, all but two mutations were within 6 bases of the AID hotspot motif, WRCH (SEQ ID NO: 5)/DGYW (SEQ ID NO: 6), strongly suggesting that the mutations arose due to SHM (Table 1A hereinbelow).

TABLE 1A
SHM-generated mutations on hT27 TCR by sample.

| | | DNA | Freq. | Mutated | CCS | Codon | Protein | | |
|---|---|---|---|---|---|---|---|---|---|
| Sample: | α/β: | Mutation: | (%): | reads: | reads: | change: | change: | Domain: | Notes: |
| h5 | WT control - no mutations found | | | | 18366 | | | | |
| h5 | α | G374C | 20.6% | 4017 | 19456 | GGC → GCC | G125A | Jα | |
| MHA | α | G374T | 8.8% | 1705 | 19456 | GGC → GTC | G125V | Jα | |
| h5 HA | β | G326A | 99.6% | 14650 | 14702 | AGT → AAT | S109N | CDR3β | |
| h5 HA | β | G326A | 99.2% | 13315 | 13422 | AGT → AAT | S109N | CDR3β | |
| HiEx | | | | | | | | | |
| h7 HA | β | G326A | 87.8% | 13953 | 15899 | AGT → AAT | S109N | CDR3β | |
| | α | A565G | 100.0% | 15919 | 15926 | AGC → GGC | S189G | Cα | |
| h7 HA | β | G326A | 56.4% | 7966 | 14125 | AGT → AAT | S109N | CDR3β | Mutations |
| HiEx | α | G164T | 26.3% | 3272 | 12444 | TGG → TTA | W55L + | FR2α | on FR2α |
| | α | G165A | 28.4% | 4023 | 14174 | | Y56F | | and CDR3β |
| | α | A167T | 27.7% | 3901 | 14067 | TAC → TTC | | | were mutually |
| | α | A565G | 99.9% | 14153 | 14164 | AGC → GGC | S189G | Cα | exclusive |
| h8 | β | C188T | 14.0% | 2269 | 16152 | ACC → ATT | T63I | Adjacent | C189T alone |
| MHA | β | C189T | 17.3% | 2819 | 16251 | | | to CDR2β | is silent |
| | β | C795T | 21.4% | 3460 | 16203 | TAC → TAT | silent | Cb | |
| | α | G374C | 14.8% | 2410 | 16268 | GGC → GCC | G125A | Jα | |
| | α | G374T | 19.2% | 3130 | 16268 | GGC → GTC | G125V | Jα | |
| h8 HA | β | G326A | 99.7% | 15790 | 15841 | AGT → AAT | S109N | CDR3β | |
| h8 HA | β | G95C | 38.8% | 5091 | 13115 | AGC → ACC | S32T | FR1β | |
| HiEx | β | G326A | 99.5% | 13495 | 13565 | AGT → AAT | S109N | CDR3β | |
| h12 | β | G326A | 57.3% | 9948 | 17374 | AGT → AAT | S109N | CDR3β | |
| MHA | | | | | | | | | |
| h12 | β | G326A | 65.2% | 15 | 23 | AGT → AAT | S109N | CDR3β | Sanger |
| HA* | | | | | | | | | shows ≈100% |
| | | | | | | | | | mutated |
| h12 HA | β | G326A | 99.9% | 9469 | 9474 | AGT → AAT | S109N | CDR3β | |
| HiEx | | | | | | | | | |

Mutations in less than 3% were considered background and are not listed.
CCS = circular consensus reads generated from multiple passes of the same molecule to generate a more accurate sequence. Domains: C = constant, CDR = complementary determining region, FR = framework region, J = joining.
*Sample with too few reads to rely on a high throughput sequencing technology.

TABLE 1B
SHM-generated mutations on hT27 TCR by mutation.

| | DNA | Codon | Protein | | | | Freq. | bp from | bp from |
|---|---|---|---|---|---|---|---|---|---|
| α/β: | Mutation: | change: | change: | m#: | Domain: | Samples: | (%): | AID hotspot: | WA: |
| β | G95C | AGC → | S32T | m1 | FR1β | h.8 HA HiEx | 38.8% | 6 | 6 |
| | | ACC | | | | | | | |
| β | G326A | AGT → | S109N | m2 | CDR3β | All except h.5 | 57-99.9% | 2 | 1 |
| | | AAT | | | | MHA + h.8 MHA | | | |
| β | C188T | ACC → | T63I | m3 | Adjacent | h.8 MHA | 14.0% | 10 | 6 |
| β | C189T | ATT | | | to CDR2β | | 17.3% | 11 | 5 |
| α | A565G | AGC → | S189G | m4 | Cα | h.7 HA, h.7 HA | 100, | 1 | 4 |
| | | GGC | | | | HiEx | 99.9% | | |
| α | G374C | GGC → | G125A | m7 | Jα | h.5 MHA, h.8 | 20.6, | 0 | |
| | | GCC | | | | MHA | 14.8% | | |
| α | G374T | GGC → | G125V | m8 | Jα | h.5 MHA, h.8 | 8.8, | 0 | |
| | | GTC | | | | MHA | 19.2% | | |
| α | G164T | TGG → | W55L + | m9 | FR2α | h.7 HA HiEx | 26.3% | 1 | 3 |
| α | G165A | TTA | Y56F | [m5 + m6] | | | 28.4% | 0 | |
| α | A167T | TAC → | | | | | 27.7% | 1 | 0 |
| | | TTC | | | | | | | |
| β | C795T | TAC → | silent | | Cb | h.8 MHA | 21.4% | 3 | 1 |
| | | TAT | | | | | | | |

Mutations found in multiple samples are separated by a comma, and the respective frequencies are in the same order.
Base pairs from AID hotspot indicates the distance between the mutated base and the C in a WRCH (SEQ ID NO: 5) hotspot or G in a DGYW (SEQ ID NO: 6) hotspot. If the mutation was not found exactly on an AID hotspot, the distance to the A of a WA polymerase η hotspot is presented.
Domains: C=constant, CDR=complementary determining region, FR=framework region, J=joining.
Protein mutations α W55L (m5) and α Y56F (m6) occurred as a double mutation designated m9.

### Example 2

### Activity of T Cells Genetically Engineered to Express hT27 TCRs Comprising the Identified Mutations

To analyze the effects of the identified mutations several binding and functional assays in primary T cells transduced with hT27 TCR comprising such mutations were effected.

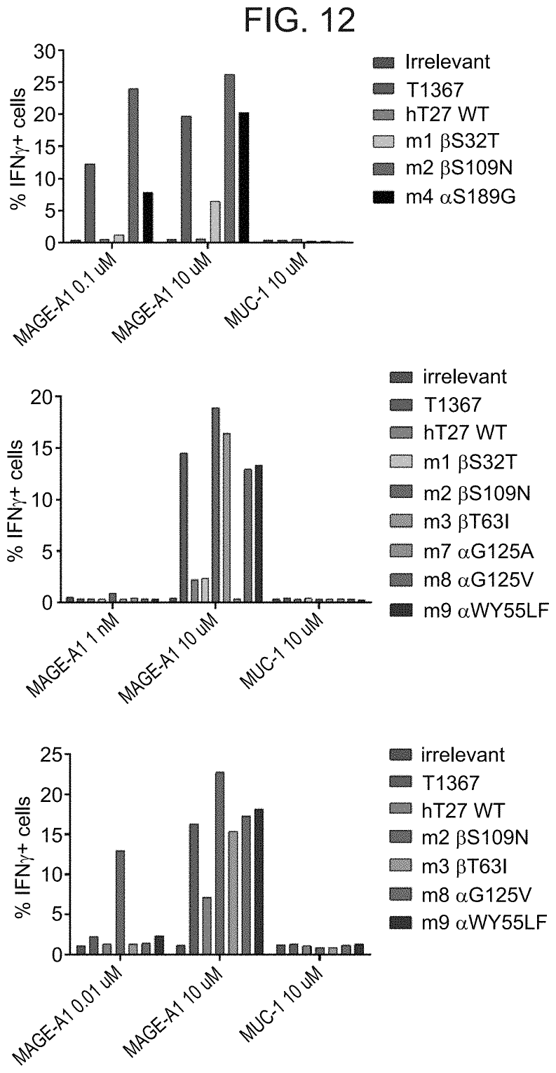

Initial screening was performed in primary mouse T cells (FIG. 12). This screening demonstrated that T cells expressing mutant hT27 TCRs comprising the identified mutations contained higher amounts of IFNγ following co-culture with T2 cells loaded with MAGE-A1 peptide, as compared to T cells expressing wild-type (WT) hT27 TCR. Of note, these levels were comparable to the IFNγ levels contained in T cells expressing the high affinity T1367 MAGE-A 1 specific TCR.

Following, analysis in human T cells from peripheral blood mononuclear cells (PMBCs) was effected on hT27 TCRs comprising S109N mutation in the beta chain (referred to herein as “m2”), T63I mutation in the beta chain (referred to herein as “m3”), S189G mutation in the alpha chain (referred to herein as “m4”), G125V mutation in the alpha chain (referred to herein as “m8”), or W55L+Y56F mutations in the alpha chain (“m9”). Of note, the transduced TCRs have a mouse TCR (mTCR) constant region, allowing for detection and gating on transduced PBMCs.

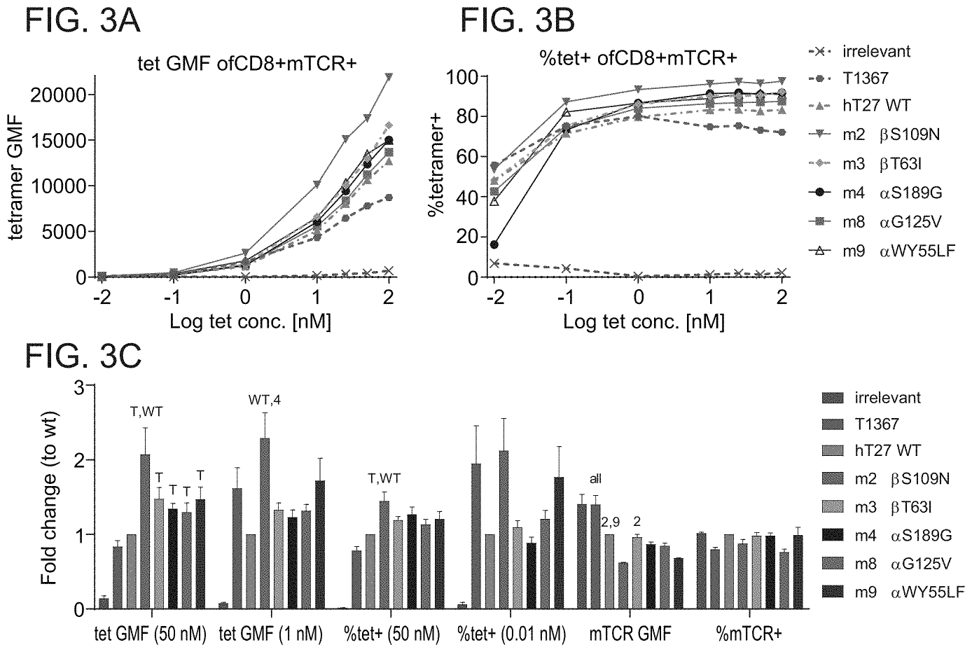

All of the mutant hT27 TCRs displayed enhanced tetramer binding compared to the WT hT27 TCR (FIGS. 3A-C). Mutant m2 was a significantly stronger binder at high and low concentrations. Interestingly, at higher tetramer concentrations all tested mutant hT27 TCRs were stronger binders than the high affinity MAGE-A1 specific T1367 TCR. At lower concentrations, m9 had comparable binding with T1367, and m3, m4, and m8 binding avidity was in between T1367 and hT27 WT (FIG. 3B). Interestingly, T1367 TCR expression level was significantly higher compared to all hT27-derived TCRs; and m2 and m9 TCRs expression levels were significantly lower compared to the WT hT27 TCR (FIG. 3C). There were no statistically significant differences in the percent of mTCR positive cells (FIG. 3C). These results show that the mutant hT27 TCRs have improved TCR binding avidity compared to the WT, which does not result from increased TCR expression.

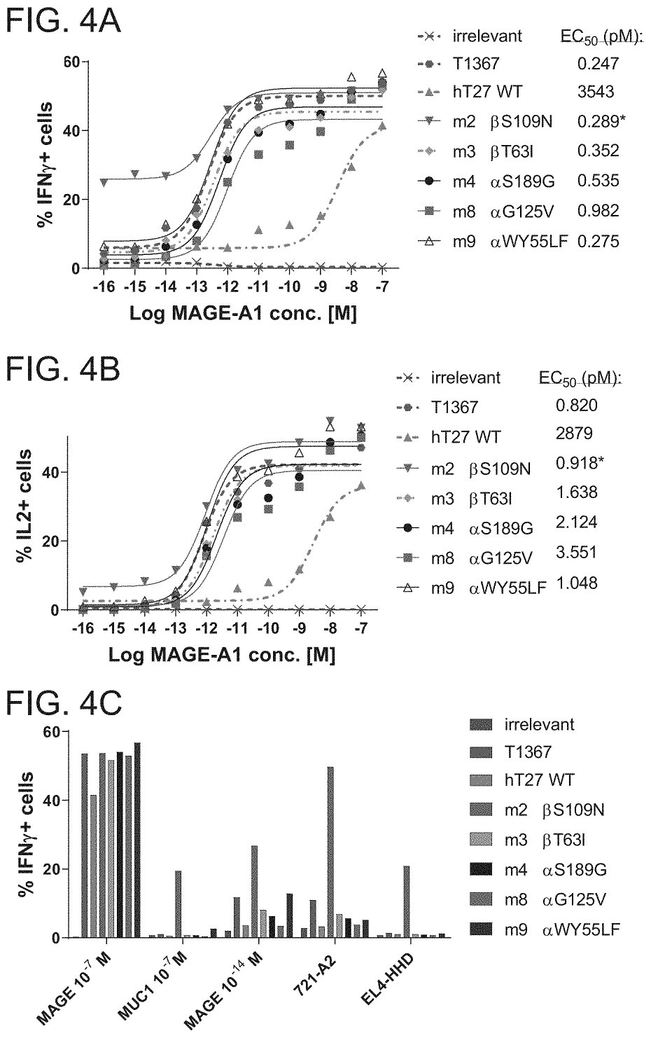

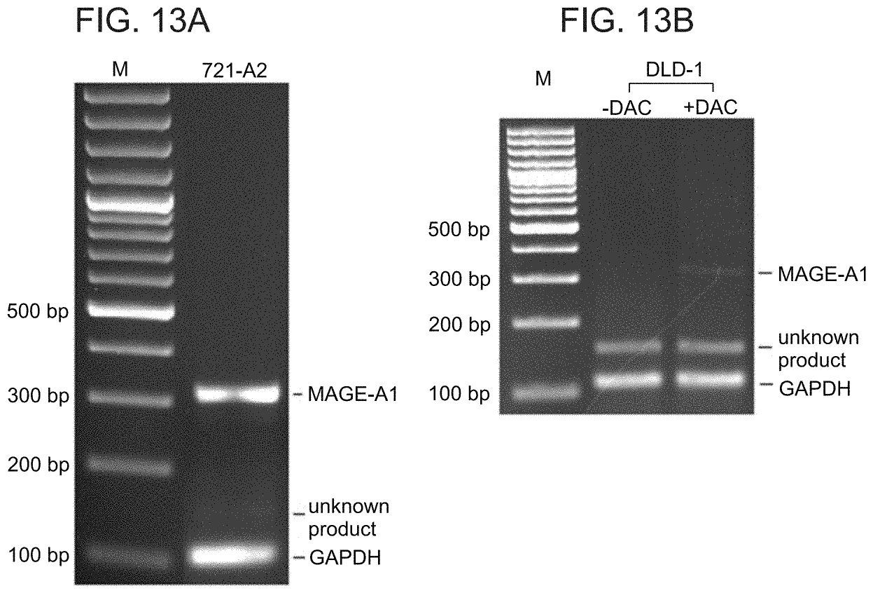

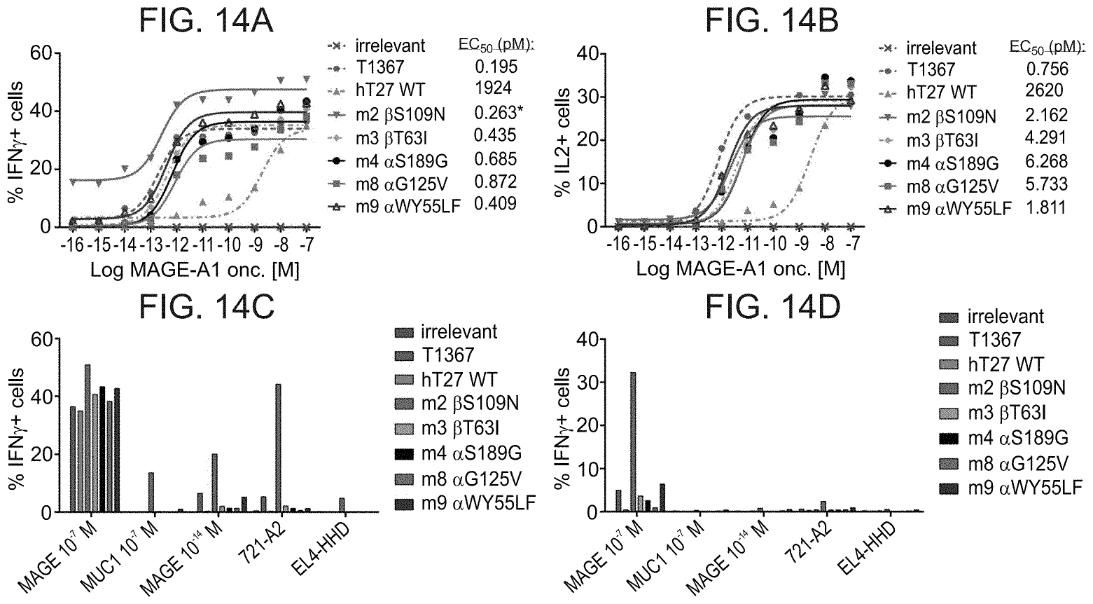

Following, intracellular cytokine staining assays for IFNγ and IL2 were effected to determine functionality of the expressed TCRs. The effective concentration 50% (EC₅₀) is peptide concentration at the halfway between maximal and minimal activity. A A low EC₅₀ indicates high functional avidity. When tested towards T2-cells expressing MAGE-A1, the EC₅₀ of the mutant hT27 TCRs was lower by approximately four orders of magnitude for IFNγ production (FIG. 4A) and three orders of magnitude for IL2 production compared to the WT hT27 TCR (FIG. 4B). It should be noted that although m2 clearly had the strongest response, the EC₅₀ is misleading because the minimum activity was much higher than the others. Hence, the order of functional avidity of IFNγ production (FIG. 4A) was m2 (EC₅₀=0.289 but not representative)>T1367 (EC₅₀=0.247 pM)>m9 (0.275 pM)>m3 (0.352 pM)>m4 (0.535 pM)>m8 (0.982 pM)>>WT hT27 (3543 pM). This trend was the same with IL2 (FIG. 4B). The order of activity towards 721.211-A2 cells expressing MAGE-A1 (FIG. 13A) was m2 (49.7% IFNγ+)>>T1367 (11%)>m3 (6.9%)>m4 (5.6%)>m9 (5.4%)>m8 (3.8%)>WT (3.3%) (FIG. 4C. Of note, all TCRs except for m2 did not show non-specific activity towards T2 cells with an irrelevant peptide, MUC1₁₃₋₂₁, or EL4-HHD cells, which don't express human MAGE-A1 (FIG. 4C). Further, all of the trends in cytokine production were also observed in PBMCs obtained from a second donor (FIGS. 14A-D). Interestingly, m2 had reduced non-specific activity in CD4+ cells (FIG. 14D), as has been shown with other ultra-high avidity TCRs (7).

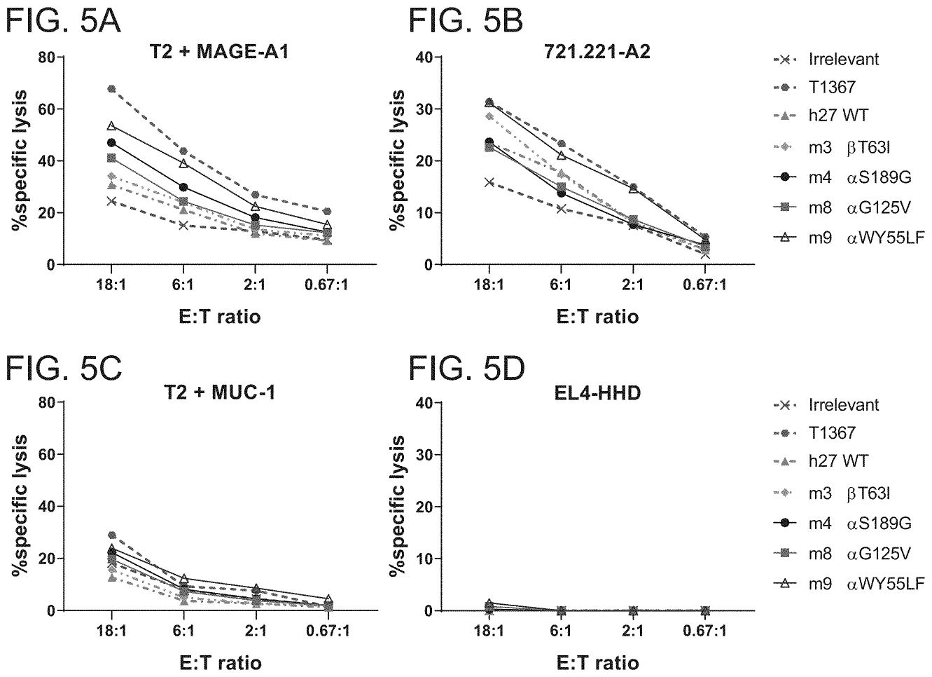

In the next step, the cytotoxic activity of the PBMCs expressing the mutant hT27 TCR towards target cell was evaluated. When tested towards T2 cells loaded with MAGE-A1₂₇₈₋₂₈₆ peptide, the order of cytotoxic activity was T1367>m9>m4>m8>m3>hT27 WT (FIG. 5A). When tested towards 721.211-A2 cells the order of activity was T1367, m9>m3>m4, m8, hT27 WT (FIG. 5B). No non-specific killing above background was observed towards T2 cells loaded with an irrelevant peptide, MUC1₁₃₋₂₁ (FIG. 5C), or EL4-HHD cells (FIG. 5D).

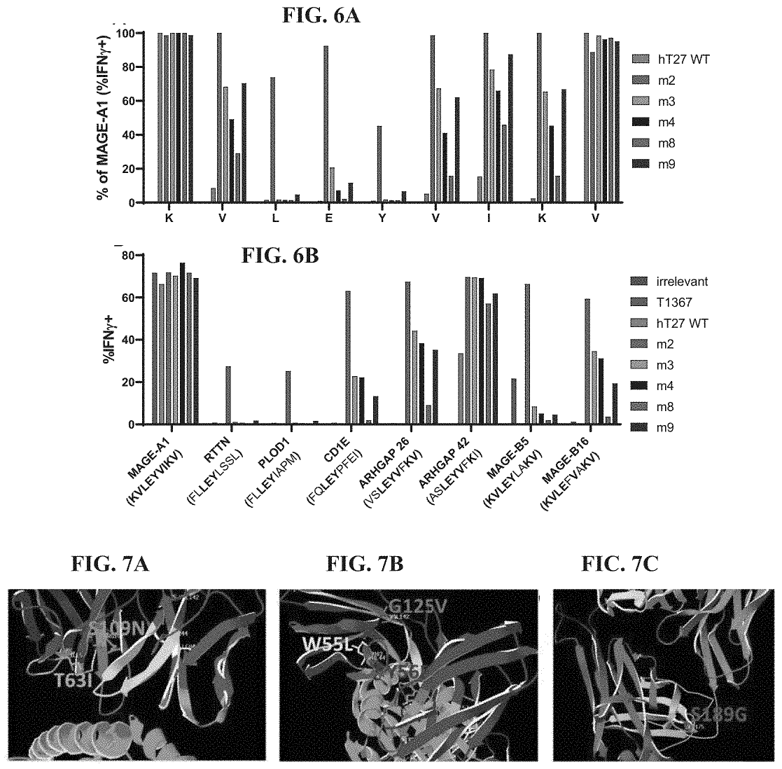

To further understand the effects of the mutations of TCR specificity d an alanine screening assay in transduced PBMCs was performed (FIG. 6A). A substitution which leads to a significant reduction can be viewed as part of the recognition motif of the TCR. The observed recognition motif of the mutant TCRs m3, m4, m8, and m9 was xxLEYxxxx (SEQ ID NO: 35). For m2 there was a strong reaction regardless of substitutions. For the WT hT27 TCR it is difficult to define a motif because there was a substantial reduction with substitutions at the tested peptide concentration in all positions except for p1 and p9. However, it should be noted that changes to the sequence xxLEYVxKx (SEQ ID NO: 36) completely abrogated activity. In the human proteome there are 171 peptides containing the xxLEYxxxx (SEQ ID NO: 35) motif that are predicted to bind HLA-A2 (as determined by the FuzzPro algorithm as described in the Materials and Methods section hereinabove, data not shown). Cross-reactivity screening was evaluated to the three strongest predicted binders containing the xxLEYxxxx (SEQ ID NO: 35) motif (RTTN, PLOD1, and CD1E), two peptides that contained the xxLEYVxKx (SEQ ID NO: 36) sequence (ARHGAP26 and 42) and two highly similar peptides from MAGE-B5 and B16 (FIG. 6B). The WT hT27 TCR only displayed cross-reactivity towards ARHGAP42. Mutant m2 reacted with all peptides. Mutants m3, m4, m8, and m9 reacted strongly to ARHGAP42 and moderately to ARHGAP26, MAGE-B5 and 16, and CD1E. T1367 only reacted moderately to MAGE-B5, however the recognition motif of T1367 is xxxEYxIKx (SEQ ID NO: 37) (10), which is not found in any of these peptides. Mutant TCRs m3, m4, m8, m9 are less sensitive to substitutions than the WT TCR and have a degree of increased cross-reactivity. However, they have not lost specificity and did not respond to EL4-HHD cells (FIGS. 4C and 5D), which express numerous peptides containing the xxLEYxxxx (SEQ ID NO: 35) motif (38) (according to RNAseq data from GSM 3022105), including many that are in the human proteome (as determined by the FuzzPro algorithm as described in the Materials and Methods section hereinabove, data not shown).

Taken together, the generated mutant hT27 TCRs displayed enhanced avidity and activity compared to the WT hT27 TCR.

### Example 3

### Structural Modeling of hT27 TCRs Comprising the Identified Mutations

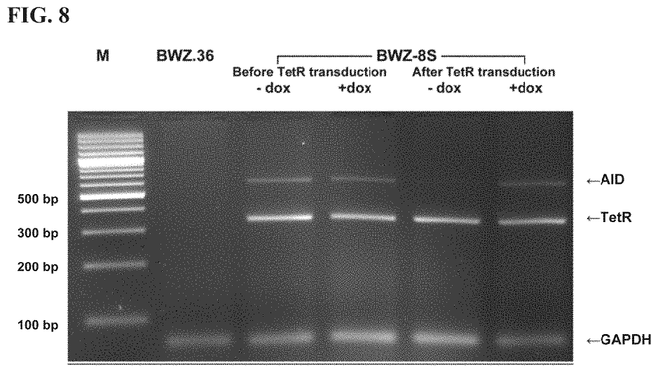

To further understand how the SHM-generated mutations influence avidity and specificity, structural modeling was effected. TCRmodel (29) was used to model the variable regions and mutations m2, m3, m8, and m9 were simulated. The numbering of amino acids on the TCR sequence and the model differ. Mutation m2 is in the stem of the CDR3β loop (FIG. 7A); mutation m3 is in the stem immediately before the CDR2β loop (FIG. 7A); mutation m8, α G125V, is in the hinge between the CDR3α loop and strand of the joining region (FIG. 7B); and the mutations in m9 are closely following the CDR1α loop and they also interact with the stem of the CDR3α loop and core of the alpha chain (FIG. 7B). All of these mutations may influence the flexibility/conformation of the CDR loops, among additional potential effects. The examined TCR contains murine constant regions, so it was able to analyze the known structure of the mouse 2C TCR (39) and simulate the m4 mutation. Mutation m4, a S189N (the equivalent of 175 on the 2C TCR), is in the DE loop of the Cα domain (FIG. 7C), which interacts with CD3 (40).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. A. W. Goldrath, M. J. Bevan, Selecting and maintaining a diverse T-cell repertoire. Nature 402, 255-62 (1999).
2. S. a Rosenberg, N. P. Restifo, J. C. Yang, R. a Morgan, M. E. Dudley, Adoptive cell transfer: a clinical path to effective cancer immunotherapy. Nat. Rev. Cancer 8, 299-308 (2008).
3. M. H. Kershaw, J. A. Westwood, C. Y. Slaney, P. K. Darcy, Clinical application of genetically modified T cells in cancer therapy. Clin. Transl. Immunol. 3, e16 (2014).
4. O. Michielin, et al., Delimiting Maximal CD8 T Cell Function Evidence for a TCR Affinity Threshold Evidence for a TCR Affinity Threshold Delimiting Maximal CD8 T Cell Function. J. Immunol. 184, 4936-4946 (2010).
5. M. Irving, et al., Interplay between T cell receptor binding kinetics and the level of cognate peptide presented by major histocompatibility complexes governs CD8+ T cell responsiveness. J. Biol. Chem. 287, 23068-78 (2012).
6. P. D. Holler, L. K. Chlewicki, D. M. Kranz, TCRs with high affinity for foreign pMHC show self-reactivity. Nat. Immunol. 4, 55-62 (2003).
7. Y. Zhao, et al., High-Affinity TCRs Generated by Phage Display Provide CD4+ T Cells with the Ability to Recognize and Kill Tumor Cell Lines. J. Immunol. 179, 5845-5854 (2007).
8. K. A. Hogquist, T. A. Baldwin, S. C. Jameson, Central tolerance: learning self-control in the thymus. Nat. Rev. Immunol. 5, 772-782 (2005).
9. M. Aleksic, et al., Different affinity windows for virus and cancer-specific T-cell receptors: implications for therapeutic strategies. Eur. J. Immunol. 42, 3174-9 (2012).
10. M. Obenaus, et al., Identification of human T-cell receptors with optimal affinity to cancer antigens using antigen-negative humanized mice. Nat. Biotechnol. 33, 402-7 (2015).
11. Y. Li, et al., Directed evolution of human T-cell receptors with picomolar affinities by phage display. Nat. Biotechnol. 23, 349-54 (2005).
12. P. D. Holler, et al., In vitro evolution of a T cell receptor with high affinity for peptide/MHC. Proc. Natl. Acad. Sci. U.S.A 97, 5387-92 (2000).
13. A. S. Chervin, D. H. Aggen, J. M. Raseman, D. M. Kranz, Engineering higher affinity T cell receptors using a T cell display system. J. Immunol. Methods 339, 175-84 (2008).
14. D. Bassan, Y. M. Gozlan, A. Sharbi-Yunger, E. Tzehoval, L. Eisenbach, Optimizing T cell receptor avidity with somatic hypermutation. Int. J. Cancer, ijc.32612 (2019).
15. Z. Luo, D. Ronai, M. D. Scharff, The role of activation-induced cytidine deaminase in antibody diversification, immunodeficiency, and B-cell malignancies. J. Allergy Clin. Immunol. 114, 726-35; quiz 736 (2004).
16. I. B. Rogozin, M. Diaz, Cutting edge: DGYW/WRCH is a better predictor of mutability at G:C bases in Ig hypermutation than the widely accepted RGYW/WRCY motif and probably reflects a two-step activation-induced cytidine deaminase-triggered process. J. Immunol. 172, 3382-4 (2004).
17. P. F. Robbins, et al., Tumor regression in patients with metastatic synovial cell sarcoma and melanoma using genetically engineered lymphocytes reactive with NY-ESO-1. J. Clin. Oncol. 29, 917-24 (2011).
18. A. P. Rapoport, et al., NY-ESO-1-specific TCR-engineered T cells mediate sustained antigen-specific antitumor effects in myeloma. Nat. Med. 21, 914-921 (2015).
19. G. P. Linette, et al., Cardiovascular toxicity and titin cross-reactivity of affinity-enhanced T cells in myeloma and melanoma. Blood 122, 863-71 (2013).
20. R. A. Morgan, et al., Cancer Regression and Neurological Toxicity Following Anti-MAGE-A3 TCR Gene Therapy. J. Immunother. 36, 133-151 (2013).
21. J. L. Weon, P. R. Potts, The MAGE protein family and cancer. Curr. Opin. Cell Biol. 37, 1-8 (2015).
22. S. Ottaviani, Y. Zhang, T. Boon, P. van der Bruggen, A MAGE-1 antigenic peptide recognized by human cytolytic T lymphocytes on HLA-A2 tumor cells. Cancer Immunol. Immunother. 54, 1214-1220 (2005).
23. S. Sanderson, N. Shastri, LacZ inducible, antigen/MHC-specific T cell hybrids. Int. Immunol. 6, 369-376 (1994).
24. S. P. Kerkar, et al., Genetic engineering of murine CD8+ and CD4+ T cells for preclinical adoptive immunotherapy studies. J. Immunother. 34, 343-52 (2011).
25. M. Wang, Z. Yang, C. Rada, M. S. Neuberger, AID upmutants isolated using a high-throughput screen highlight the immunity/cancer balance limiting DNA deaminase activity. Nat. Struct. Mol. Biol. 16, 769-776 (2009).
26. F. Van Den Ent, J. Löwe, RF cloning: A restriction-free method for inserting target genes into plasmids. J. Biochem. Biophys. Methods 67, 67-74 (2006).
27. Y. Akatsuka, et al., Efficient cloning and expression of HLA class I cDNA in human B-lymphoblastoid cell lines. Tissue Antigens 59, 502-511 (2002).
28. S. Pascolo, et al., HLA-A2.1-restricted Education and Cytolytic Activity of CD8+ T Lymphocytes from β2 Microglobulin (β2m) HLA-A2.1 Monochain Transgenic H-2db β2m Double Knockout Mice. J. Exp. Med. 185, 2043-2051 (1997).
29. R. Gowthaman, B. G. Pierce, TCRmodel: high resolution modeling of T cell receptors from sequence. Nucleic Acids Res. 46, W396-W401 (2018).
30. G. Cafri, A. Sharbi-Yunger, E. Tzehoval, L. Eisenbach, Production of LacZ inducible T cell hybridoma specific for human and mouse gp100₂₅₋₃₃ peptides. PLoS One 8, e55583 (2013).
31. N.-Y. Zheng, K. Wilson, M. Jared, P. C. Wilson, Intricate targeting of immunoglobulin somatic hypermutation maximizes the efficiency of affinity maturation. J. Exp. Med. 201 (2005).
32. I. B. Rogozin, Y. I. Pavlov, K. Bebenek, T. Matsuda, T. A. Kunkel, Somatic mutation hotspots correlate with DNA polymerase |[eta]| error spectrum. Nat. Immunol. 2, 530-536 (2001).
33. Y. I. Pavlov, et al., Correlation of somatic hypermutation specificity and A-T base pair substitution errors by DNA polymerase eta during copying of a mouse immunoglobulin kappa light chain transgene. Proc. Natl. Acad. Sci. U.S.A 99, 9954-9 (2002).
34. N. Michael, et al., The E Box Motif CAGGTG Enhances Somatic Hypermutation without Enhancing Transcription. Immunity 19, 235-242 (2003).
35. A. Tanaka, H. M. Shen, S. Ratnam, P. Kodgire, U. Storb, Attracting AID to targets of somatic hypermutation. J. Exp. Med. 207, 405-415 (2010).
36. K. B. J. Scholten, et al., Codon modification of T cell receptors allows enhanced functional expression in transgenic human T cells. Clin. Immunol. 119, 135-45 (2006).
37. A. Rhoads, K. F. Au, PacBio Sequencing and Its Applications. Genomics. Proteomics Bioinformatics 13, 278-289 (2015).
38. C. M. Laumont, et al., Noncoding regions are the main source of targetable tumor-specific antigens. Sci. Transl. Med. 10 (2018).
39. K. C. Garcia, et al., T cell receptor interactions with syngeneic and allogeneic ligands: Affinity measurements and crystallization. Proc. Natl. Acad. Sci. 94, 13838-13843 (1997).
40. M. S. Kuhns, M. M. Davis, Disruption of Extracellular Interactions Impairs T Cell Receptor-CD3 Complex Stability and Signaling. Immunity 26, 357-369 (2007).
41. P. F. Robbins, et al., Single and Dual Amino Acid Substitutions in TCR CDRs Can Enhance Antigen-Specific T Cell Functions. J. Immunol. 180, 6116-6131 (2008).
42. P. Sharma, D. M. Kranz, Subtle changes at the variable domain interface of the T-cell receptor can strongly increase affinity. J. Biol. Chem., jbc.M117.814152 (2017).
43. K. Malecek, et al., Engineering improved T cell receptors using an alanine-scan guided T cell display selection system. J. Immunol. Methods 392, 1-11 (2013).
44. R. Ohta, A. Demachi-Okamura, Y. Akatsuka, H. Fujiwara, K. Kuzushima, Improving TCR affinity on 293T cells. J. Immunol. Methods 466, 1-8 (2019).
45. X. Liu, et al., Affinity-Tuned ErbB2 or EGFR Chimeric Antigen Receptor T Cells Exhibit an Increased Therapeutic Index against Tumors in Mice. Cancer Res. 75, 3596-3607 (2015).

What is claimed is:

1. A T cell receptor (TCR) comprising a TCR α chain as set forth in SEQ ID NO: 1 having at least one mutation at an amino acid position selected from the group consisting of S189, G125, W55 and Y56; and/or a TCR β chain as set forth in SEQ ID NO: 2 having at least one mutation at an amino acid position selected from the group consisting of S32, S109 and T63, the TCR binds a MAGE-A1 peptide as set forth in SEQ ID NO: 25.

2. The TCR of claim 1, wherein said mutation in S189 comprises an S189G, said mutation in G125 comprises a G125A or G125V, said mutation in W55 comprises a W55L, said mutation in Y56 comprises a Y56F, said mutation in S32 comprises a S32T, said mutation in S109 comprises a S109N and/or said mutation in T63 comprises a T631.

3. The TCR of claim 1, wherein said TCR comprises:
(i) a TCR α chain as set forth in SEQ ID NO: 1 and a TCR β chain as set forth in SEQ ID NO: 2 having S32T and S109N mutations;
(ii) a TCR α chain as set forth in SEQ ID NO: 1 and a TCR β chain as set forth in SEQ ID NO: 2 having a S109N mutation;
(iii) a TCR α chain as set forth in SEQ ID NO: 1 and a TCR β chain as set forth in SEQ ID NO: 2 having a T63I mutation;
(iv) a TCR α chain as set forth in SEQ ID NO: 1 having a S189G mutation and a TCR β chain as set forth in SEQ ID NO: 2;
(v) a TCR α chain as set forth in SEQ ID NO: 1 having a G125A mutation and a TCR β chain as set forth in SEQ ID NO: 2;
(vi) a TCR α chain as set forth in SEQ ID NO: 1 having a G125V mutation and a TCR β chain as set forth in SEQ ID NO: 2;
(vii) a TCR α chain as set forth in SEQ ID NO: 1 having W55L and Y56F mutations and a TCR β chain as set forth in SEQ ID NO: 2;
(viii) a TCR α chain as set forth in SEQ ID NO: 1 having W55L, Y56F and S189G mutations and a TCR β chain as set forth in SEQ ID NO: 2; or
(ix) a TCR α chain as set forth in SEQ ID NO: 1 having a S189G mutation and a TCR β chain as set forth in SEQ ID NO: 2 having a S109N mutation.

4. The TCR of claim 1, having increased avidity to said MAGE-A1 peptide as compared to a TCR having a TCR α chain as set forth in SEQ ID NO: 1 and a TCR β chain as set forth in SEQ ID NO: 2.

5. A T cell receptor (TCR) comprising:
(i) a mutation at a constant region of a TCR α chain at an amino acid position S71 corresponding to SEQ ID NO: 38;
(ii) at least one mutation at a V region of a TCR α chain at an amino acid position selected from the group consisting of W55 and Y56 corresponding to SEQ ID NO: 39, wherein said TCR α chain comprises a TRaV5 V region;
(iii) at least one mutation at a J region of a TCR α chain at an amino acid position G12 corresponding to SEQ ID NO: 40, wherein said TCR α chain comprises a TRaJ34 J region; and/or
(iv) at least one mutation at a V region of a TCR β chain at an amino acid position selected from the group consisting of S32 and T63 corresponding to SEQ ID NO: 41, wherein said TCR β chain comprises a TRbV20-1 V region.

6. The TCR of claim 5, wherein said mutation in S71 comprises an S71G, said mutation in G12 comprises a G12A or G12V, said mutation in W55 comprises a W55L, said mutation in Y56 comprises a Y56F, said mutation in S32 comprises a S32T and/or said mutation in T63 comprises a T63I.

7. The TCR of claim 5, wherein said TCR binds a tumor associated antigen (TAA).

8. The TCR of claim 5, wherein said TCR binds a MAGE-A1 peptide as set forth in SEQ ID NO: 25.

9. At least one polynucleotide encoding the TCR of claim 1.

10. A T cell genetically engineered to express the TCR of claim 1.

11. At least one polynucleotide encoding the TCR of claim 5.

12. A T cell genetically engineered to express the TCR of claim 5.

13. A method of treating cancer presenting a MAGE-A1 peptide as set forth in SEQ ID NO: 25 in a subject in need thereof, the method comprising administering to the subject a therapeutically effective amount of a T cells genetically engineered to express the TCR of claim 1, thereby treating the cancer in the subject.

14. A method of treating cancer presenting a MAGE-A1 peptide as set forth in SEQ ID NO: 25 in a subject in need thereof, the method comprising administering to the subject a therapeutically effective amount of a T cells genetically engineered to express the TCR of claim 8, thereby treating the cancer in the subject.

15. A method of treating a disease that can benefit from adoptive transfer of T cells in a subject in need thereof, the method comprising administering to the subject a therapeutically effective amount of T cells genetically engineered to express the TCR of claim 5, wherein pathologic cells of said subject present a peptide identified by said TCR, thereby treating the disease in the subject.

16. The method of claim 15, wherein said disease is cancer.

17. A method for modulating the avidity of a T cell receptor (TCR) to its ligand, the method comprising:
(a) expressing in a T cell a nucleic acid sequence encoding the TCR, said nucleic acid sequence has been codon optimized to:
(i) maximize the number of WRCH (SEQ ID NO: 5) or DGYW (SEQ ID NO: 6) nucleic acid sequences,
(ii) minimize the number of SYC or GRS nucleic acid sequences,
(iii) maximize the number of WA nucleic acid sequences and/or
(iv) minimize the number of rare codons; and
(b) expressing in said T cell Activation Induced cytidine Deaminase (AID) having an amino acid sequence as set forth in SEQ ID NO: 7.

18. A method for modulating the avidity of a or a Chimeric antigen receptor (CAR) to its ligand, the method comprising:
(a) expressing in a cell a nucleic acid sequence encoding the CAR; and
(b) expressing in said cell Activation Induced cytidine Deaminase (AID).

19. The method of claim 18, wherein said nucleic acid sequence expressed in said (a) has been codon optimized to:
(i) maximize the number of WRCH (SEQ ID NO: 5) or DGYW (SEQ ID NO: 6) nucleic acid sequences,
(ii) minimize the number of SYC or GRS nucleic acid sequences,
(iii) maximize the number of WA nucleic acid sequences and/or
(iv) minimize the number of rare codons.

20. The method of claim 18, wherein said cell is a T cell.


SEQUENCE LISTING (60 sequences)

SEQ ID NO: 1 - protein, 269 aa, synthetic construct
  REGION 1..269: note = hT27 TCR alpha chain aa sequence
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAGSGGGTDKLIFGTGTRLQVFPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 2 - protein, 304 aa, synthetic construct
  REGION 1..304: note = hT27 TCR beta chain aa sequence
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAREPGQGPYEQYFGPGTRLTVTEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 3 - DNA, 807 bp, synthetic construct
  misc_feature 1..807: note = hT27 TCR alpha chain NA sequence
  source 1..807: mol_type = other DNA
atgaagaccttcgccggcttcagcttcctgttcctgtggctgcagctggactgcatgagcagaggcgaggacgtggaacagagcctgtttctgagcgtgcgcgagggcgacagcagcgtgatcaattgcacctacaccgactccagcagcacctacctgtactggtacaagcaggaacccggcgctggcctgcagctgctgacctacatcttcagcaacatggacatgaagcaggaccagagactgaccgtgctgctgaacaagaaggacaagcacctgagcctgcggatcgccgatacccagacaggcgacagcgccatctacttttgtgctggtagcggcggcggtaccgataaacttatatttggcaccggcaccagactgcaggtgttccccaacatccagaaccccgagcccgccgtgtaccagctgaaggaccctagaagccaggacagcaccctgtgcctgttcaccgacttcgacagccagatcaacgtgcccaagaccatggaaagcggcaccttcatcaccgacaagacagtgctggatatgaaggccatggacagcaagagcaacggcgccattgcctggtccaaccagaccagcttcacatgccaggacatcttcaaagagacaaacgccacctaccccagcagcgacgtgccctgtgatgccaccctgaccgagaagtccttcgagacagacatgaacctgaacttccagaacctgagcgtgatgggcctgagaatcctgctgctgaaggtggccggcttcaacctgctgatgaccctgagactgtggtccagc

SEQ ID NO: 4 - DNA, 912 bp, synthetic construct
  misc_feature 1..912: note = hT27 TCR beta chain NA sequence
  source 1..912: mol_type = other DNA
atgcttttgttgctgctgctgctgggccctggctctggactgggagctgtggtgtctcagcacccctcctgggtcatctgcaagagcggcaccagcgtgaagatcgagtgcagaagcctggacttccaggccaccaccatgttctggtacagacagttccccaagcagagcctgatgctgatggccacctccaacgagggcagcaaggccacatatgagcagggcgtggaaaaggacaagttcctgatcaaccacgccagcctgaccctgagcaccctgacagtgacaagcgcccaccccgaggacagcagcttctacatctgtagtgcacgcgaacctgggcaaggaccttacgaacagtactttggccctggcacccggctgaccgtgaccgaggatctgagaaacgtgaccccccctaaggtgtccctgttcgagcctagcaaggccgagatcgccaacaagcagaaagccaccctcgtgtgcctggccagaggcttcttccccgaccacgtggaactgagttggtgggtcaacggcaaagaggtgcacagcggcgtgtccaccgatccccaggcctacaaagagagcaactacagctactgcctgagcagcagactgcgggtgtccgccaccttctggcacaacccccggaaccacttcagatgccaggtgcagtttcacggcctgagcgaagaggacaagtggcccgagggctcccctaagcccgtgacccagaatatctctgccgaagcctggggcagagccgactgtggcattaccagcgccagctaccatcagggcgtgctgagcgccaccatcctgtacgagatcctgctgggaaaggccaccctgtacgccgtgctggtgtcaggactggtgctgatggctatggtcaagaagaagaactcc

SEQ ID NO: 5-6 - skipped (2 entries)

SEQ ID NO: 7 - protein, 198 aa, synthetic construct
  REGION 1..198: note = human mutant AID 7.3 AA
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTLGL

SEQ ID NO: 8 - DNA, 597 bp, synthetic construct
  misc_feature 1..597: note = human mutant AID 7.3 NA
  source 1..597: mol_type = other DNA
atggacagcctcttgatgaaccggagggagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcatctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacggaagaactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 9 - DNA, 22 bp, synthetic construct
  misc_feature 1..22: note = Single strand DNA oligonucleotide
  source 1..22: mol_type = other DNA
atttgtctgaaaattagctcga

SEQ ID NO: 10 - DNA, 20 bp, synthetic construct
  misc_feature 1..20: note = Single strand DNA oligonucleotide
  source 1..20: mol_type = other DNA
agagcaactacagctactgc

SEQ ID NO: 11 - DNA, 24 bp, synthetic construct
  misc_feature 1..24: note = Single strand DNA oligonucleotide
  source 1..24: mol_type = other DNA
catttaaatgtatacccaaatcaa

SEQ ID NO: 12 - DNA, 20 bp, synthetic construct
  misc_feature 1..20: note = Single strand DNA oligonucleotide
  source 1..20: mol_type = other DNA
cctcaaagtagacggcatcg

SEQ ID NO: 13 - DNA, 18 bp, synthetic construct
  misc_feature 1..18: note = Single strand DNA oligonucleotide
  source 1..18: mol_type = other DNA
atggacagcctcttgatg

SEQ ID NO: 14 - DNA, 18 bp, synthetic construct
  misc_feature 1..18: note = Single strand DNA oligonucleotide
  source 1..18: mol_type = other DNA
tcaaagtcccaaagtacg

SEQ ID NO: 15 - DNA, 18 bp, synthetic construct
  misc_feature 1..18: note = Single strand DNA oligonucleotide
  source 1..18: mol_type = other DNA
cgtaaactcgcccagaag

SEQ ID NO: 16 - DNA, 19 bp, synthetic construct
  misc_feature 1..19: note = Single strand DNA oligonucleotide
  source 1..19: mol_type = other DNA
agtaaaatgccccacagcg

SEQ ID NO: 17 - DNA, 20 bp, synthetic construct
  misc_feature 1..20: note = Single strand DNA oligonucleotide
  source 1..20: mol_type = other DNA
cgtgttcctacccccaatgt

SEQ ID NO: 18 - DNA, 24 bp, synthetic construct
  misc_feature 1..24: note = Single strand DNA oligonucleotide
  source 1..24: mol_type = other DNA
tgtcatcatacttggcaggtttct

SEQ ID NO: 19 - DNA, 21 bp, synthetic construct
  misc_feature 1..21: note = Single strand DNA oligonucleotide
  source 1..21: mol_type = other DNA
caacttcactcgacagaggca

SEQ ID NO: 20 - DNA, 20 bp, synthetic construct
  misc_feature 1..20: note = Single strand DNA oligonucleotide
  source 1..20: mol_type = other DNA
cctaggcaggtgacaaggac

SEQ ID NO: 21 - DNA, 21 bp, synthetic construct
  misc_feature 1..21: note = Single strand DNA oligonucleotide
  source 1..21: mol_type = other DNA
tcaccagggctgcttttaact

SEQ ID NO: 22 - DNA, 22 bp, synthetic construct
  misc_feature 1..22: note = Single strand DNA oligonucleotide
  source 1..22: mol_type = other DNA
gccatgggtggaatcatattgg

SEQ ID NO: 23 - DNA, 49 bp, synthetic construct
  misc_feature 1..49: note = Single strand DNA oligonucleotide
  misc_feature 21..28: note = n is a, c, g, or t
  source 1..49: mol_type = other DNA
gactgtacagtgatcgtacgnnnnnnnntccaagctcacttacaggcgg

SEQ ID NO: 24 - DNA, 50 bp, synthetic construct
  misc_feature 1..50: note = Single strand DNA oligonucleotide
  misc_feature 21..28: note = n is a, c, g, or t
  source 1..50: mol_type = other DNA
ctgatcgatcgtcaactagcnnnnnnnntggcggtaagatgctcgaattc

SEQ ID NO: 25 - protein, 9 aa, synthetic construct
  REGION 1..9: note = MAGE-A1278-286 peptide
KVLEYVIKV

SEQ ID NO: 26 - protein, 9 aa, synthetic construct
  REGION 1..9: note = MUC113-21 peptide
LLLTVLTVV

SEQ ID NO: 27 - skipped

SEQ ID NO: 28 - protein, 773 aa, synthetic construct
  REGION 1..773: note = CD3 AA sequence (construct)
MEHSGILASLILIAVLPQGSPFKIQVTEYEDKVFVTCNTSVMHLDGTVEGWFAKNKTLNLGKGVLDPRGIYLCNGTEQLAKVVSSVQVHYRMCQNCVELDSGTMAGVIFIDLIATLLLALGIYCFAGHETGRPSGAAEVQALLKNEQLYQPLRDREDTQYSRLGGNWPRNKKSGPVKQTLNFDLLKLAGDVESNPGPMEQRKGLAGLFLVISLLQGTVAQTNKAKNLVQVDGSRGDGSVLLTCGLTDKTIKWLKDGSIISPLNATKNTWNLGNNAKDPRGTYQCQGAKETSNPLQVYYRMCENCIELNIGTISGFIFAEVISIFFLALGVYLIAGQDGVRQSRASDKQTLLQNEQLYQPLKDREYDQYSHLQGNQLRKKRSEGRGSLLTCGDVEENPGPMRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAVGSATNFSLLKQAGDVEENPGPMKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 29 - DNA, 39 bp, synthetic construct
  misc_feature 1..39: note = CDR3alpha na before optimization
  source 1..39: mol_type = other DNA
tgcgccggctctggcggcggaaccgacaagctgatcttt

SEQ ID NO: 30 - protein, 13 aa, synthetic construct
  REGION 1..13: note = CDR3alpha na before optimization
CAGSGGGTDKLIF

SEQ ID NO: 31 - DNA, 39 bp, synthetic construct
  misc_feature 1..39: note = CDR3alpha na following optimization
  source 1..39: mol_type = other DNA
tgtgctggtagcggcggcggtaccgataaacttatattt

SEQ ID NO: 32 - DNA, 45 bp, synthetic construct
  misc_feature 1..45: note = CDR3beta na before optimization
  source 1..45: mol_type = other DNA
tgcagcgccagagagcccggccagggaccttacgagcagtacttt

SEQ ID NO: 33 - protein, 15 aa, synthetic construct
  REGION 1..15: note = CDR3beta aa
CSAREPGQGPYEQYF

SEQ ID NO: 34 - DNA, 45 bp, synthetic construct
  misc_feature 1..45: note = CDR3beta na following optimization
  source 1..45: mol_type = other DNA
tgtagtgcacgcgaacctgggcaaggaccttacgaacagtacttt

SEQ ID NO: 35 - skipped

SEQ ID NO: 36 - protein, 9 aa, synthetic construct
  REGION 1..9: note = Recognition motif
  REGION 1..2: note = misc_feature - Xaa can be any naturally occurring amino acid
  SITE 7: note = misc_feature - Xaa can be any naturally occurring amino acid
  SITE 9: note = misc_feature - Xaa can be any naturally occurring amino acid
XXLEYVXKX

SEQ ID NO: 37 - protein, 9 aa, synthetic construct
  REGION 1..9: note = Recognition motif
  REGION 1..3: note = misc_feature - Xaa can be any naturally occurring amino acid
  SITE 6: note = misc_feature - Xaa can be any naturally occurring amino acid
  SITE 9: note = misc_feature - Xaa can be any naturally occurring amino acid
XXXEYXIKX

SEQ ID NO: 38 - protein, 138 aa, synthetic construct
  REGION 1..138: note = mouse TCR alpha constant region (TRaC) aa sequence
PNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 39 - protein, 113 aa, synthetic construct
  REGION 1..113: note = TCR alpha TRaV5 V region aa sequence
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAGS

SEQ ID NO: 40 - protein, 19 aa, synthetic construct
  REGION 1..19: note = TCR alpha TRaV5 V region aa sequence
SYNTDKLIFGTGTRLQVFP

SEQ ID NO: 41 - protein, 111 aa, synthetic construct
  REGION 1..111: note = TCR beta TRbV20-1 V region aa sequence
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAR

SEQ ID NO: 42 - protein, 5 aa, synthetic construct
  REGION 1..5: note = TCR beta TRbV20-1 V region aa sequence
EPGQG

SEQ ID NO: 43 - protein, 15 aa, synthetic construct
  REGION 1..15: note = TCR beta TRbJ2-7 J region aa sequence
SYEQYFGPGTRLTVT

SEQ ID NO: 44 - protein, 173 aa, synthetic construct
  REGION 1..173: note = mouse TCR beta constant region (TRbC2) aa sequence
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 45 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 46 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 47 - protein, 127 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 48 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 49 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 51 - protein, 19 aa, synthetic construct
  REGION 1..19: note = TCR alpha TRaJ34 J region aa sequence in hT27
GGGTDKLIFGTGTRLQVFP

SEQ ID NO: 52 - protein, 140 aa, synthetic construct
  REGION 1..140: note = TCR alpha TRaJ34 J region aa sequence in hT27
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 53 - protein, 15 aa, synthetic construct
  REGION 1..15: note = TCR beta TRbJ2-7 J region aa sequence in hT27
PYEQYFGPGTRLTVT

SEQ ID NO: 54 - DNA, 912 bp, synthetic construct
  misc_feature 1..912: note = TCR beta TRbJ2-7 J region aa sequence in hT27
  source 1..912: mol_type = other DNA
atgcttttgttgctgctgctgctgggccctggctctggactgggagctgtggtgtctcagcacccctcctgggtcatctgcaagagcggcaccaccgtgaagatcgagtgcagaagcctggacttccaggccaccaccatgttctggtacagacagttccccaagcagagcctgatgctgatggccacctccaacgagggcagcaaggccacatatgagcagggcgtggaaaaggacaagttcctgatcaaccacgccagcctgaccctgagcaccctgacagtgacaagcgcccaccccgaggacagcagcttctacatctgtagtgcacgcgaacctgggcaaggaccttacgaacagtactttggccctggcacccggctgaccgtgaccgaggatctgagaaacgtgaccccccctaaggtgtccctgttcgagcctagcaaggccgagatcgccaacaagcagaaagccaccctcgtgtgcctggccagaggcttcttccccgaccacgtggaactgagttggtgggtcaacggcaaagaggtgcacagcggcgtgtccaccgatccccaggcctacaaagagagcaactacagctactgcctgagcagcagactgcgggtgtccgccaccttctggcacaacccccggaaccacttcagatgccaggtgcagtttcacggcctgagcgaagaggacaagtggcccgagggctcccctaagcccgtgacccagaatatctctgccgaagcctggggcagagccgactgtggcattaccagcgccagctaccatcagggcgtgctgagcgccaccatcctgtacgagatcctgctgggaaaggccaccctgtacgccgtgctggtgtcaggactggtgctgatggctatggtcaagaagaagaactcc

SEQ ID NO: 55 - DNA, 912 bp, synthetic construct
  misc_feature 1..912: note = hT27 mutant m2 beta chain Na sequence
  source 1..912: mol_type = other DNA
atgcttttgttgctgctgctgctgggccctggctctggactgggagctgtggtgtctcagcacccctcctgggtcatctgcaagagcggcaccagcgtgaagatcgagtgcagaagcctggacttccaggccaccaccatgttctggtacagacagttccccaagcagagcctgatgctgatggccacctccaacgagggcagcaaggccacatatgagcagggcgtggaaaaggacaagttcctgatcaaccacgccagcctgaccctgagcaccctgacagtgacaagcgcccaccccgaggacagcagcttctacatctgtaatgcacgcgaacctgggcaaggaccttacgaacagtactttggccctggcacccggctgaccgtgaccgaggatctgagaaacgtgaccccccctaaggtgtccctgttcgagcctagcaaggccgagatcgccaacaagcagaaagccaccctcgtgtgcctggccagaggcttcttccccgaccacgtggaactgagttggtgggtcaacggcaaagaggtgcacagcggcgtgtccaccgatccccaggcctacaaagagagcaactacagctactgcctgagcagcagactgcgggtgtccgccaccttctggcacaacccccggaaccacttcagatgccaggtgcagtttcacggcctgagcgaagaggacaagtggcccgagggctcccctaagcccgtgacccagaatatctctgccgaagcctggggcagagccgactgtggcattaccagcgccagctaccatcagggcgtgctgagcgccaccatcctgtacgagatcctgctgggaaaggccaccctgtacgccgtgctggtgtcaggactggtgctgatggctatggtcaagaagaagaactcc

SEQ ID NO: 56 - DNA, 912 bp, synthetic construct
  misc_feature 1..912: note = hT27 mutant m3 beta chain Na sequence
  source 1..912: mol_type = other DNA
atgcttttgttgctgctgctgctgggccctggctctggactgggagctgtggtgtctcagcacccctcctgggtcatctgcaagagcggcaccagcgtgaagatcgagtgcagaagcctggacttccaggccaccaccatgttctggtacagacagttccccaagcagagcctgatgctgatggccatttccaacgagggcagcaaggccacatatgagcagggcgtggaaaaggacaagttcctgatcaaccacgccagcctgaccctgagcaccctgacagtgacaagcgcccaccccgaggacagcagcttctacatctgtagtgcacgcgaacctgggcaaggaccttacgaacagtactttggccctggcacccggctgaccgtgaccgaggatctgagaaacgtgaccccccctaaggtgtccctgttcgagcctagcaaggccgagatcgccaacaagcagaaagccaccctcgtgtgcctggccagaggcttcttccccgaccacgtggaactgagttggtgggtcaacggcaaagaggtgcacagcggcgtgtccaccgatccccaggcctacaaagagagcaactacagctactgcctgagcagcagactgcgggtgtccgccaccttctggcacaacccccggaaccacttcagatgccaggtgcagtttcacggcctgagcgaagaggacaagtggcccgagggctcccctaagcccgtgacccagaatatctctgccgaagcctggggcagagccgactgtggcattaccagcgccagctaccatcagggcgtgctgagcgccaccatcctgtacgagatcctgctgggaaaggccaccctgtacgccgtgctggtgtcaggactggtgctgatggctatggtcaagaagaagaactcc

SEQ ID NO: 57 - DNA, 807 bp, synthetic construct
  misc_feature 1..807: note = hT27 mutant m4 alpha chain Na sequence
  source 1..807: mol_type = other DNA
atgaagaccttcgccggcttcagcttcctgttcctgtggctgcagctggactgcatgagcagaggcgaggacgtggaacagagcctgtttctgagcgtgcgcgagggcgacagcagcgtgatcaattgcacctacaccgactccagcagcacctacctgtactggtacaagcaggaacccggcgctggcctgcagctgctgacctacatcttcagcaacatggacatgaagcaggaccagagactgaccgtgctgctgaacaagaaggacaagcacctgagcctgcggatcgccgatacccagacaggcgacagcgccatctacttttgtgctggtagcggcggcggtaccgataaacttatatttggcaccggcaccagactgcaggtgttccccaacatccagaaccccgagcccgccgtgtaccagctgaaggaccctagaagccaggacagcaccctgtgcctgttcaccgacttcgacagccagatcaacgtgcccaagaccatggaaagcggcaccttcatcaccgacaagacagtgctggatatgaaggccatggacggcaagagcaacggcgccattgcctggtccaaccagaccagcttcacatgccaggacatcttcaaagagacaaacgccacctaccccagcagcgacgtgccctgtgatgccaccctgaccgagaagtccttcgagacagacatgaacctgaacttccagaacctgagcgtgatgggcctgagaatcctgctgctgaaggtggccggcttcaacctgctgatgaccctgagactgtggtccagc

SEQ ID NO: 58 - DNA, 807 bp, synthetic construct
  misc_feature 1..807: note = hT27 mutant m7 alpha chain Na sequence
  source 1..807: mol_type = other DNA
atgaagaccttcgccggcttcagcttcctgttcctgtggctgcagctggactgcatgagcagaggcgaggacgtggaacagagcctgtttctgagcgtgcgcgagggcgacagcagcgtgatcaattgcacctacaccgactccagcagcacctacctgtactggtacaagcaggaacccggcgctggcctgcagctgctgacctacatcttcagcaacatggacatgaagcaggaccagagactgaccgtgctgctgaacaagaaggacaagcacctgagcctgcggatcgccgatacccagacaggcgacagcgccatctacttttgtgctggtagcggcggcggtaccgataaacttatatttggcaccgccaccagactgcaggtgttccccaacatccagaaccccgagcccgccgtgtaccagctgaaggaccctagaagccaggacagcaccctgtgcctgttcaccgacttcgacagccagatcaacgtgcccaagaccatggaaagcggcaccttcatcaccgacaagacagtgctggatatgaaggccatggacagcaagagcaacggcgccattgcctggtccaaccagaccagcttcacatgccaggacatcttcaaagagacaaacgccacctaccccagcagcgacgtgccctgtgatgccaccctgaccgagaagtccttcgagacagacatgaacctgaacttccagaacctgagcgtgatgggcctgagaatcctgctgctgaaggtggccggcttcaacctgctgatgaccctgagactgtggtccagc

SEQ ID NO: 59 - DNA, 807 bp, synthetic construct
  misc_feature 1..807: note = hT27 mutant m8 alpha chain Na sequence
  source 1..807: mol_type = other DNA
atgaagaccttcgccggcttcagcttcctgttcctgtggctgcagctggactgcatgagcagaggcgaggacgtggaacagagcctgtttctgagcgtgcgcgagggcgacagcagcgtgatcaattgcacctacaccgactccagcagcacctacctgtactggtacaagcaggaacccggcgctggcctgcagctgctgacctacatcttcagcaacatggacatgaagcaggaccagagactgaccgtgctgctgaacaagaaggacaagcacctgagcctgcggatcgccgatacccagacaggcgacagcgccatctacttttgtgctggtagcggcggcggtaccgataaacttatatttggcaccgtcaccagactgcaggtgttccccaacatccagaaccccgagcccgccgtgtaccagctgaaggaccctagaagccaggacagcaccctgtgcctgttcaccgacttcgacagccagatcaacgtgcccaagaccatggaaagcggcaccttcatcaccgacaagacagtgctggatatgaaggccatggacagcaagagcaacggcgccattgcctggtccaaccagaccagcttcacatgccaggacatcttcaaagagacaaacgccacctaccccagcagcgacgtgccctgtgatgccaccctgaccgagaagtccttcgagacagacatgaacctgaacttccagaacctgagcgtgatgggcctgagaatcctgctgctgaaggtggccggcttcaacctgctgatgaccctgagactgtggtccagc

SEQ ID NO: 60 - DNA, 807 bp, synthetic construct
  misc_feature 1..807: note = hT27 mutant m9 alpha chain Na sequence
  source 1..807: mol_type = other DNA
atgaagaccttcgccggcttcagcttcctgttcctgtggctgcagctggactgcatgagcagaggcgaggacgtggaacagagcctgtttctgagcgtgcgcgagggcgacagcagcgtgatcaattgcacctacaccgactccagcagcacctacctgtacttattcaagcaggaacccggcgctggcctgcagctgctgacctacatcttcagcaacatggacatgaagcaggaccagagactgaccgtgctgctgaacaagaaggacaagcacctgagcctgcggatcgccgatacccagacaggcgacagcgccatctacttttgtgctggtagcggcggcggtaccgataaacttatatttggcaccggcaccagactgcaggtgttccccaacatccagaaccccgagcccgccgtgtaccagctgaaggaccctagaagccaggacagcaccctgtgcctgttcaccgacttcgacagccagatcaacgtgcccaagaccatggaaagcggcaccttcatcaccgacaagacagtgctggatatgaaggccatggacagcaagagcaacggcgccattgcctggtccaaccagaccagcttcacatgccaggacatcttcaaagagacaaacgccacctaccccagcagcgacgtgccctgtgatgccaccctgaccgagaagtccttcgagacagacatgaacctgaacttccagaacctgagcgtgatgggcctgagaatcctgctgctgaaggtggccggcttcaacctgctgatgaccctgagactgtggtccagc